(19) United States
(12) Patent Application Publication
(10) Pub. No.: US 2020/0197294 A1
(43) Pub. Date: Jun. 25, 2020

(54) NOVEL CANNABIS LINES AND EXTRACTS FOR SKIN REJUVENATION AND SKIN PROTECTION

(71) Applicants: Pathway RX Inc., Lethbridge (CA); Inplanta Biotechnology Inc., Lethbridge (CA)

(72) Inventors: Olga Kovalchuk, Lethbridge (CA); Dongping Li, Lethbridge (CA); Rocio del Carmen Rodriguez-Juarez, Lethbridge (CA); Anna Kovalchuk, Lethbridge (CA); Dwight Darryl Hudson, Raymond (CA); Igor Kovalchuk, Lethbridge (CA)

(21) Appl. No.: 16/711,665

(22) Filed: Dec. 12, 2019

Related U.S. Application Data
(60) Provisional application No. 62/781,633, filed on Dec. 19, 2018.

(51) Int. Cl. A61K 8/9789; A61K 8/34; A61K 8/49; A61K 8/06; A61K 8/04; A61Q 19/08

(52) U.S. Cl. CPC A61K 8/9789; A61K 8/347; A61Q 19/08; A61K 8/06; A61K 8/044; A61K 8/498

(57) ABSTRACT

The present invention provides new Cannabis lines, extracts and methods for skin rejuvenation and healing and protection against harmful environmental factors such as UV rays and other damaging agents. The method includes generation of unique lines, whole plant extract preparation, exposing human 3D skin tissues to UV and treating human 3D skin tissues with extracts in amount sufficient to modulate gene expression in the skin tissues before and after UV exposure. The modulation of gene expression then results in a reduction of the disease state-associated changes or aspects thereof in the cannabis-treated skin tissues.

### FIELD OF THE INVENTION

The present invention relates generally to products and methods for treating skin, and more specifically to methods and products for treating skin from cannabis and hemp plants.

### BACKGROUND OF THE INVENTION

Skin is the largest organ in the body covering a surface area of about 1.8 m² and accounting for about 8% of the total body mass. Skin functions as a barrier, preventing pathogens and toxicants from entering into the body and as a regulator for water and heat balance.

Skin consists of dermal and epidermal tissues. Epidermis, the outermost layer of the skin, is composed of a basal layer, which is located at the bottom of epidermis and a suprabasal layer, which is located at the upper part of epidermis. The suprabasal layer contains three sub-layers—spinous, granular, and cornified layers. Keratinocytes are the major cellular component of the epidermal tissue (over 95%). Other cell types are fibroblasts, melanocytes, Langerhans cells, and others[1].

Aging is biologically determined and environmentally modified process of loss of viability and increase in vulnerability is associated with a gradual loss of homeostatic mechanisms that maintain the structure and function of adult tissues. It is associated with an increased risk of several morbidities such cancer, cardiovascular disease, and autoimmune disease. It is associated with the body's altered capacity to cope with stress induced by metabolism, infection, and damage to cellular macromolecules and tissues. In skin, aging is the most visible.

The clinical manifestations of skin aging are fine wrinkles, thin and transparent skin, loss of underlying fat leading to hollowed cheeks and eye sockets, dry and itchy skin, lack of sufficient perspiration, hair graying, hair loss or hirsutism, and thinning of the nail plates [2].

Skin aging can be intrinsic or extrinsic. Intrinsic is largely genetically determined via mutations, hormonal deregulation, and altered cellular metabolism. With time, it leads to decreased proliferative capacity, cellular senescence, and altered biosynthetic activity. Extrinsic aging occurs as results of exposure to environmental insults such as sun UV radiation, tobacco smoking, and exposure to extreme temperature, air pollution and toxic chemicals, stress, lack of sleep and poor nutrition. Of those, UV exposure is the most common, very potent and extensively studies. Extrinsic aging factors can promote intrinsically-determined aging processes. Both intrinsic and extrinsic aging are linked to inflammation, cytokine deregulation, oxidative stress, mitochondrial function loss/mutations, altered DNA repair and stability, cell cycle and apoptosis, and overall cellular metabolism.

Chronologically aged and photo-aged skin share important molecular features including decreased proliferative capacity of skin-derived cells, decreased matrix synthesis in the dermis associated with an increased expression of enzymes that degrade the collagenous matrix, increased inflammatory process and oxidative stress [3, 4]. The extracellular matrix (ECM) components such as elastin, fibrillin, and collagens are significantly degenerated in the aged skin [5]. Collagen is responsible for skin's firmness and strength. In skin, collagen is the most abundant protein, accounting for 80% of its content. While type I and III collages are prevalent in skin, and especially in the dermal layer, other collagens (types V, VI, VII, XI and others) are also found in varying amounts [6].

Collagen fibrils in skin are formed by collagens type I and III. Reduced expression and synthesis of collagen types I and III is characteristic of chronologically aged skin as well as photoaged skin [7]. Recent studies show that skin aging is characterized by marked decrease in the expression levels of COL1A1, COL1A2, COL3A1, COL5A1, COL5A2 [8].

Type VII collagen forms anchoring fibrils at the dermal-epidermal junction. The decrease collagen VII weakens linkage between dermis and epidermis and thus leads to development of skin wrinkles [5]. Collagen VII levels also decrease with aging and upon UV exposure.

Elastin, another ECM component, allows skin to stretch and contract, supports its tightness ad structure. Elastin levels decrease with age [6]. Skin aging is also linked to a decrease in the levels of large chondroitin sulfate proteoglycan (versican) [9]. Furthermore, heparan sulfate proteoglycan (HSPG) or perlecan is important for tissue organization and structural integrity [10], and its levels decrease with age.

Tenascin-X regulates both the structure and stability of elastic fibers and organizes collagen fibrils in the extra-cellular matrix (ECM), impacting the rigidity or elasticity of virtually every cell in the body [11]. Skin aging is also characterized by increases in inflammation and is termed “inflammaging” [5].

In previous studies, aged skin fibroblasts exhibited decreased expression of genes and proteins involved in extracellular matrix structure (COL3A1, COL1A1, BGN), cell cycle regulation, cellular antioxidant defenses. Simultaneously, genes and proteins involved in inflammation processes (CCL3, CCLS, CCR2, S100A), apoptosis regulation (BAX), and extracellular matrix degradation (MMP1, MMP3) and others were significantly elevated [8].

Matrix metalloproteinases (MMPs) are important for remodeling of the extracellular matrix. UV was shown to cause upregulation of MMPs that partake in destruction of type I and III fibrillary collagen in the derma, as well as degradation of non-collagen components of the derma, including glycoproteins and basement membrane proteoglycans.

Skin aging and skin inflammation are key targets of cosmetic and pharmaceutical industries [5, 6, 8], and there is a huge need in novel, safe and effective anti-aging modalities. With degrease in the ozone layer and increase in the UV levels on the planet, there is a need for new, safe and effective UV protection and healing skin modalities.

As the average age of the people on earth is increasing, there is an increased unmet need for effective skin treatment compositions and methods.

### SUMMARY OF THE INVENTION

It is an object of some aspects of the present invention, to provide improved methods and products for treating, improving and curing skin ageing.

It is another object of some aspects of the present invention, to provide improved methods and products for treating, improving and curing skin ailments.

It is an object of some aspects of the present invention to provide compositions for improving wellness in a human or mammalian organism.

It is another object of some aspects of the present invention to provide compositions for preventing or treating diseases or disorders in a human or mammalian organism.

The compositions and dosage forms of the present invention are useful in promoting health and preventing or treating a large number of disorders in human patients and other mammalian subjects.

In additional embodiments of the present invention, compositions and methods are provided for treating and/or preventing skin aging and disorders.

The present invention is directed to compositions and methods for treating disorders, in general, and more particularly, skin aging and skin diseases/disorders. The compositions of the present invention may be used for improving wellness of a human or mammalian subject. Additionally, the compositions of the present invention may be used to treat any disorder or ailment in a human patient or mammalian subject. Furthermore, the compositions of the present invention may conveniently used in conjunction with a drug to treat any disorder or ailment in a human patient or mammalian subject.

In additional embodiments of the present invention, compositions and methods are provided for treating and/or preventing proliferative disorders.

In additional embodiments of the present invention, compositions and methods are provided for treating and/or preventing cancer.

In some embodiments of the present invention, improved methods and products are provided for skin treatment. In other embodiments of the present invention, a method and product is described for improving skin appearance.

In other embodiments of the present invention, a method and product is described for protecting skin from UV radiation.

In other embodiments of the present invention, a method and product is described for healing skin after UV radiation exposure.

Some embodiments of the present invention provide compounds, compositions and formulations from at least one of hemp and cannabis.

Some further embodiments of the present invention provide methods for upregulating at least one collagen pathway gene.

Some further embodiments of the present invention provide methods for upregulating at least one elastin gene.

Some further embodiments of the present invention provide methods for upregulating expression of at least one retinol metabolism pathway gene.

Some further embodiments of the present invention provide methods for downregulating expression of at least one interleukin gene.

Some further embodiments of the present invention provide methods for downregulating expression of at least one inflammatory pathway gene.

Some further embodiments of the present invention provide methods for downregulating expression of at least TNF gene.

Some further embodiments of the present invention provide methods for downregulating expression of at least MMP gene.

Some further embodiments of the present invention provide methods for downregulating expression of at least S100A family gene.

Some further embodiments of the present invention provide methods for upregulating at least one collagen pathway gene product.

Some further embodiments of the present invention provide methods for upregulating at least one retinol metabolism pathway product.

Some further embodiments of the present invention provide methods for downregulating at least one interleukin gene product.

Some further embodiments of the present invention provide methods for downregulating at least one inflammatory pathway gene product.

Some further embodiments of the present invention provide methods for downregulating at least TNF gene product.

Some further embodiments of the present invention provide methods for downregulating at least MMP gene product.

Some further embodiments of the present invention provide methods for downregulating at least S100 A gene product. Some further embodiments of the present invention provide methods for downregulating at least SPRR gene product.

Some further embodiments of the present invention provide methods for downregulating at least serpin gene product.

In other embodiments of the present invention, a method and product is described for improving skin tension and elasticity.

In other embodiments of the present invention, a method and product is described for improving skin appearance.

In other embodiments of the present invention, a method and product is described for improving skin health.

In other embodiments of the present invention, a method and product is described for reducing skin age.

In additional embodiments for the present invention, new Cannabis sativa lines are provided.

In additional embodiments for the present invention, new extracts from new Cannabis sativa lines are provided.

In additional embodiments for the present invention, new extracts from new Cannabis sativa lines are provided, having anti-aging activity.

The present invention further provides new unique cannabis lines, dried powders from the extracts, compositions comprising the powders or parts thereof, compounds derived therefrom, pharmaceutical compositions comprising the compound(s), extracts and methods for skin rejuvenation and healing and protection against harmful environmental factors such as UV rays and other damaging agents. The method includes generation and characterization of unique lines, whole plant extract preparation, exposing human 3D skin tissues to UV and treating human 3D skin tissues with extracts in amount sufficient to modulate gene expression in the skin tissues before and after UV exposure. The modulation of gene expression then results in a reduction of the disease state-associated changes or aspects thereof in the cannabis-treated skin tissues.

The present invention provides new Cannabis sativa lines, cultivars and extracts and method of using them as a means to modulate gene expression in normal human skin tissues and skin tissues exposed to UV. The disclosure provides methods of modulating gene expression through the application cannabis extracts to normal skin.

The disclosure also provides methods of modulating gene expression through the application cannabis extracts to skin, affected by UV exposure, to prevent or mitigate UV-induced photo-aging.

Accordingly, the present invention provides a means for modulating gene expression by providing extracts of new cannabis lines to skin cells or skin tissue, before and after UV exposure, in an amount sufficient to modulate gene expression where modulation of gene expression results in a reduction of aging markers, UV protection and post-UV healing.

The present invention provides freshly prepared extracts of twenty two new C. sativa lines and identified six lines with the best anti-aging (#4, 13, 6, 5, 39 and 273) properties, UV healing (#4, 13, 6) and protection properties (#15).

Using EpiDermFT human 3D skin tissue models exposed to UV and then treated with extracts of new cannabis lines via their addition to the tissue growth media it was demonstrated that 5 new extracts strongly upregulate collagen gene expression, and strongly down-regulate expression of inflammation, immunity and ECM-related genes that are involved in skin aging and skin diseases (lines #4, #15, #13, #6, #12).

Using EpiDermFT human 3D skin tissue models treated with extracts of new cannabis lines and then exposed to UV, it was shown that one new C. sativa line demonstrates UV protection properties that manifest via altered expression of collagen genes and genes involved in inflammation, extracellular matrix (ECM) and immunity (line #15).

Using EpiDermFT human 3D skin tissue models topically treated with extracts of new cannabis lines dissolved in coconut oil, it was shown that three lines (#5, #39 and #273) demonstrate excellent anti-aging properties that manifest via elevated expression of numerous collagen and elastin genes and decreased expression of genes involved in inflammation.

Using EpiDermFT human 3D skin tissue models topically treated with extracts of new cannabis lines dissolved in coconut oil, it was shown that two lines (#31 and #166) demonstrate anti-aging properties that manifest via elevated expression of COL1A1 gene and decreased expression of genes involved in inflammation.

In various embodiments, at least one of the deregulated genes is selected from the group consisting of Epidermal Differentiation Complex (EDC) genes, collagen genes, elastin genes, genes involved in modulation of inflammation, oxidative stress, immunity and autoimmunity, cellular matrix, cellular proliferation and apoptosis.

In particular, there are described methods for preparing compositions, compounds, formulations and extracts for treating a skin disorder or disease, or skin aging, in a human patient.

There is thus provided according to some embodiments of the present invention, a composition, derived from at least one of hemp and cannabis for treating a skin disorder or disease, or skin aging, in a human patient.

A use of a solvent extract from at least one of hemp and cannabis, according to some embodiments of the present invention, is for the manufacture of a pharmaceutical composition for the treatment of a skin disease or disorder or skin aging.

Some embodiments of the present invention are directed to a method for treating skin in a human patient comprising administering to said patient a pharmaceutically effective amount of the cannabis extract composition as described herein.

Additionally, some further embodiments of the present invention are directed to a method for treating a skin disorder or disease in a human patient comprising administering to said patient the oral dosage form as described herein.

The liquid cannabis extracts of the present invention, and/or dry powders therefrom, are suitable for oral administration, and appear to be well absorbed through the intestine by the blood and thus exhibit the potential to heal a wide range of cancerous organs and inflammatory conditions, such as, but not limited to those mentioned by Chattopadhyay et al. Current Science 87(1) July 2004, 44-53.

According to some embodiments of the present invention, the composition or formulation further comprises at least one solvent or hydrant. In some cases, the hydrant is water, such as double-distilled water. In some cases, it may be at least one organic solvent, such as alcohol.

According to some embodiments of the present invention, the at least one solvent or hydrant is present in the composition or formulation in a concentration of 10-90%, 15-80%, 20-70%, 25-50%, 30-40%, or 10-18% by weight percent.

The solvent or hydrant may further comprise a pH regulator, such as an acid or base. In some embodiments, the base comprises sodium hydroxide.

Suitable products or compositions of the present invention may be in the form of ointments or salves, creams, emulsions, gels, foams, sprays or medicated dressings or bandages, which must be directly applied on the affected zone and must be kept in contact with the skin.

In one or more embodiments, the compositions further comprise up to 10% of water.

In one or more embodiments, the composition is substantially non-aqueous and/or substantially alcohol-free.

In another embodiment, the present invention provides a method for inhibiting a disease in a subject comprising administering a subject a composition of the invention.

In another embodiment, the present invention provides a method for inhibiting a proliferative disease in a subject comprising administering a subject a composition of the present invention.

In another embodiment, the present invention provides a method for inhibiting a disease in a subject comprising orally administering a product of the present invention to the subject.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried cannabis extracts contained therein to be orally administered without drinking.

In one or more embodiments, the composition further comprises a therapeutically effective concentration of one or more active agents.

The composition of the present invention further contains a surface-active agent. Surface-active agents (also termed “surfactants”) include any agent linking oil and water in the composition, in the form of emulsion.

In an embodiment of the present invention, a composition of the present invention includes one or more additional components. Such additional components include but are not limited to anti-static agents, buffering agents, bulking agents, chelating agents, cleansers, colorants, conditioners, diluents, dyes, emollients, fragrances, humectants, permeation enhancers, pH-adjusting agents, preservatives, protectants, skin penetration enhancers, softeners, solubilizers, sunscreens, sun blocking agents, sunless tanning agents, viscosity modifiers and vitamins As is known to one skilled in the art, in some instances a specific additional component may have more than one activity, function or effect.

The present invention demonstrate a potent anti-aging and anti-UV-induced skin damage and aging activity of novel cannabis line extracts, and may present a novel and promising natural resource for anti-aging treatments and modalities.

### NON-LIMITING EMBODIMENTS OF THE PRESENT INVENTION

1. A method for treating mammalian skin, the method comprising:
a) combining at least one marijuana cultivar/strain and at least one hemp cultivar/strain to form at least one Cannabis line;
b) extracting at least one compound from said at least one Cannabis line to form an extract; and
c) treating skin with at least one of said extract and said at least one compound in an effective amount to treat said skin.

2. A method according to embodiment 1, wherein said treating step induces modulation of gene expression in at least one of skin cells and skin tissue; and wherein said modulation of gene said expression results in a reduction of at least one of an aging state and a disease state of said skin.

3. A method according to embodiment 1, wherein said at least one Cannabis line is selected from the group consisting of a marijuana/marijuana hybrid line, hemp/hemp hybrid line and hemp/marijuana hybrid line.

4. A method according to embodiment 3, wherein said at least one line is selected from the group consisting of designated line #4,#6, #8, #12, #13, #14, #15, #18, #81, #5, #31, #39, #49, #69, #114, #166, #267, #273, #10, #11, #156, #155, #207.

5. A method according to embodiment 1, wherein said extracting step comprises extracting flowers of said at least one Cannabis line.

6. A method according to embodiment 5, wherein said extracting step comprises extracting said at least one compound in at least one organic solvent.

7. A method according to embodiment 6, wherein said extracting step is performed at a temperature in the range of 15-60 ° C. and at a pressure in a range of −0.5 to +1.5 bar and wherein said at least one organic solvent comprises ethyl acetate.

8. A method according to embodiment 1, further comprising:
i. exposing said skin to UV radiation prior to said treating step

9. A method according to embodiment 8, wherein said modulation of gene expression results in a reduction of at least one of a photo-aging state and said disease state in said skin, and wherein said skin comprises at least one of skin cells and skin tissue.

10. A method according to embodiment 1, wherein said at least one compound is provided in a concentration in a range of 0.0001-0.05 μg/μl, 0.001-0.05 μg/μl, 0.001-0.005 μg/μl, 0.003-0.03 μg/μl or 0.007-0.015 μg/μl.

11. A method according to embodiment 1, wherein said at least one compound is provided in a solvent extract and said solvent extract exhibits skin healing properties.

12. A method according to embodiment 11, wherein said solvent extract is at least 2-20, 3-15, 4-12, 5-10 or 6-9 times as effective as at least one of THC and CBD, administered at the same concentration in treating said disease.

13. The method of embodiment 2, wherein the disease state is skin cancer.

14. The method of embodiment 13, wherein the skin cancer is non-melanoma skin cancer.

15. The method of embodiment 14, wherein the non-melanoma skin cancer is a squamous cell carcinoma or basal cell carcinoma.

16. The method of embodiment 2, wherein the disease state is an inflammatory skin disease.

17. The method of embodiment 2, wherein the disease state is psoriasis or atopic dermatitis or contact dermatitis or UV or other environmental factor-induced inflammation.

18. The method of embodiment 13, wherein the skin cancer is non-melanoma skin cancer.

19. A method according to embodiment 1, wherein said Cannabis line is a Cannabis sativa line.

20. An organic extract of at least one plant line, said at least one plant line formed from combining at least one of:
a) at least one marijuana cultivar/strain; and
b) at least one hemp cultivar/strain, wherein said organic extract comprises at least one compound suitable for treating a mammalian skin disease or disorder.

21. An organic extract according to embodiment 20, wherein said at least one plant line comprises a Cannabis sativa line.

22. An organic extract according to embodiment 20, wherein said mammalian skin disease or disorder is selected from the group consisting of skin cancer, non-melanoma skin cancer, squamous cell carcinoma, basal cell carcinoma, cancer, an inflammatory skin disease, psoriasis, atopic dermatitis, contact dermatitis, a UV-induced disorder, a burn, a cut, a scar, a skin insult, or other environmental factor-induced inflammation.

23. An organic extract according to embodiment 20, wherein said extract is effective against chemo-resistant cancer cells and is suitable to overcome chemo-resistance.

24. An organic extract according to embodiment 20, wherein said extract potentiates effects of cytotoxic chemotherapy and is an effective and safe adjuvant modality.

25. An organic extract according to embodiment 20, wherein said organic extract is at least 2-20, 3-15, 4-12, 5-10 or 6-9 times as effective as at least one of THC and CBD, administered at the same concentration in treating said disease.

26. A combination therapy, isolated from an organic extract of at least one hybrid line, said at least one hybrid line formed from combining at least one of:
a) at least one marijuana cultivar/strain; and
b) at least one hemp cultivar/strain; and
   wherein said organic extract comprises a plurality of compounds suitable for treating a mammalian skin disease or disorder.

27. A combination therapy according to embodiment 26, wherein said mammalian skin disease or disorder is selected from the group consisting of skin cancer, non-melanoma skin cancer, squamous cell carcinoma, basal cell carcinoma, cancer, an inflammatory skin disease, psoriasis, atopic dermatitis, contact dermatitis, a UV-induced disorder, a burn, a cut, a scar, a skin insult, or other environmental factor-induced inflammation.

28. A line of Cannabis sativa formed by combining at least one marijuana cultivar/strain and at least one hemp cultivar/strain, said line to be deposited at publicly available culture collection under at least one designation number, specified herein as line #4, #6, #8, #12, #13, #14, #15, #18, #5, #31, #39, #49, #69, #114, #166, #267, #273, #10, #11, #156, #155, #207.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the context of this application, the word “Figure” is abbreviated to Fig.

In the drawings:

FIGS. 1A-1V show the results of high performance liquid chromatography (HPLC) profiles of tested lines, in accordance with some embodiments of the present invention;

FIG. 2 shows a scheme of the analysis flow—from new C. sativa lines towards new lines with anti-aging properties. Novel extracts were applied to the EpiDermFT tissues, and their effects on global gene expression were analyzed, in accordance with some embodiments of the present invention;

FIGS. 3A-3E show the experimental set up for EpiDermFt tissues:

FIG. 3A. EpiDermFT (Mattek) has normal skin tissue structure with differentiated dermis and epidermis and is constructed from human-derived epidermal keratinocytes and dermal fibroblasts. It exhibits in vivo-like growth and morphological characteristics whereby cells sustain differentiation and metabolic status similar to those of human epidermis. The model is widely used and accepted for platform for studying toxicity of cosmetics and topical agents, effects of ionizing radiation, skin carcinogenesis and wound healing;

FIG. 3B. Tissue insert in a well with medium;

FIG. 3C. Scheme of healing and protection experiments. For the healing experiment, tissues were exposed to UV, then treated with extracts and used for global gene expression profiling. For protection experiments, tissues were treated with extracts and then exposed to UV, and further used for gene expression profiling;

FIG. 3D. For protection experiments, tissues were treated with extracts and then exposed to UV, and further used for gene expression profiling.

FIG. 3E. To analyze the direct anti-aging effects of extracts on skin, extracts were dissolved in coconut oil and applied directly to the skin model surface; in accordance with some embodiments of the present invention;

FIG. 4A shows the effects of new cannabis lines on collagen gene expression—experiment 1. Genes with a False Discovery Rate (FDR)-adjusted p-value <0.05 and log2 fold change >0.6 (1.5× change) were considered differentially expressed. Levels of gene expression are shown as relative expression units, fold change are compared to control. Upper panel shows results of new extracts #4, 14, 15, 8, 13, 6, 12, (FIG. 4B) the lower panel focuses on the lines that caused changes in the two or more collagen genes; in accordance with some embodiments of the present invention;

FIG. 5A shows the effects of three best new cannabis lines on expression of genes involved in collagen, and extracellular matrix (ECM) maintenance—experiment 1. These lines affected expression of 3 or more collagen genes. FIG. 5B shows the effects of three best new cannabis lines on expression of genes involved in inflammation. Genes with a False Discovery Rate (FDR)-adjusted p-value <0.05 and log2 fold change >0.6 (1.5× change) were considered differentially expressed. Levels of gene expression are shown as relative expression units, fold change are compared to control in accordance with some embodiments of the present invention;

FIG. 6A shows the effects of new cannabis lines on collagen gene expression—experiment 2. Extracts were dissolved in coconut oil and applied to the surface of the skin tissues. Genes with a False Discovery Rate (FDR)-adjusted p-value <0.10 and log2 fold change >0.6 (1.5× change) were considered differentially expressed. Levels of gene expression are shown as relative expression units, fold change are compared to oil-treated control. FIG. 6A in a table form shows results of all extracts # 3, 31, 39, 49, 69, 114, 166, 267, 273, FIG. 6B shows a graph, which focuses on the lines that caused changes in the two or more collagen genes, and affected elastin, tenascin and heparan sulfate proteoglycan 2 (perlecan) genes in accordance with some embodiments of the present invention;

FIG. 7 shows the effects of new cannabis lines on expression of genes involved in skin inflammation and disease—S100A, serpins, SPRR, IL and other genes—experiment 2. Extracts were dissolved in coconut oil and applied to the surface of the skin tissues. Genes with a False Discovery Rate (FDR)-adjusted p-value <0.10 and log2 fold change >0.6 (1.5× change) were considered differentially expressed.

Levels of gene expression are shown as relative expression units, fold change are compared to oil-treated control in accordance with some embodiments of the present invention;

FIG. 8 shows the mechanisms affected by UV exposure in the UV-protection experiment by line #15, in accordance with some embodiments of the present invention; and

FIG. 9 shows the schematic summary of the main results in accordance with some embodiments of the present invention.

In all the figures similar reference numerals identify similar parts.

### DETAILED DESCRIPTION OF PREFERRED EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

The present invention further provides methods of drug discovery. According to some embodiments, the method includes:
a) combining at least one marijuana cultivar/strain and at least one hemp cultivar/strain to form at least one hybrid line;
b) extracting at least one compound from said at least one hybrid line to form an extract; and
c) testing the extract in vitro to identify a biologically active extract.

The method further includes repeating steps a) to c) on a plurality of extracts to identify the most biologically active extracts.

The method further includes isolating active compounds or components from the biologically active extracts. The method further comprises treating a patient with a disease or disorder with at least one of the active compounds, components or extracts to cure, alleviate or manage the disease or disorder.

Provided herein are new cannabis lines and their extracts and methods of their use for treating skin aging and UV-induced skin effects. The methods are exemplified, but are not limited to the steps of: 1) preparation of new cannabis extracts, 2) exposing skin tissue models to novel extracts before and after UV treatments and 3) modulating the gene expression to cause a reduction of aging/disease state, or prevent an increase in the aging state in the skin tissues.

Some additional embodiments of the present invention provide a pharmaceutical extract, pure compound, pharmaceutical composition or formulation adapted to treat, and/or protect, and/or to attenuate a skin barrier disruption in mammalian subject. The mammalian subject may be human or animal.

Some additional embodiments of the present invention provide a pharmaceutical extract, a pure compound, a formulation or composition adapted to inhibit further irritation following thermal injury, mosquito bites, abrasion, irradiation, laser, extreme low temperatures, acne, wrinkles, skin dryness, and other acute and/or chronic skin irritations involving disruption of skin barrier.

Some important embodiments of the present invention provide a pharmaceutical extract, a pure compound, a pharmaceutical composition or formulation adapted to inhibit further irritation following thermal injury, mosquito bites, abrasion, irradiation, laser, extreme low temperatures, acne, wrinkles, skin dryness, and other and/or chronic skin irritations involving disruption of skin barrier.

Additionally, some further embodiments of the present invention provide a medical device comprising a pharmaceutical composition adapted to treat, and/or protect from, a wound following thermal injury, mosquito bites, abrasion, irradiation, laser, extreme low temperatures, acne, wrinkles, skin dryness, and other and/or chronic skin irritations involving disruption of skin barrier.

A burn is an insult to the skin caused by any energy applied to the skin including heat, radiation, laser, radioactivity, extreme low temperatures, electricity, or mechanical abrasion, chafing and friction. Chemicals can also be a source of burns. Thermal, UV or other irradiation insults damage the skin through a similar pathophysiological process. Heat on skin layers can also form from skin inflammation and skin dehydration.

Although deep skin layers may be destroyed immediately following exposure to severe insults, in most cases, the stratum corneum/“skin barrier” is the first to be damaged in burns. Deeper skin layers may then be involved, because of a “domino effect”, originating from the skin barrier disruption.

The severity of a burn, or burn outcome, is a function of the intensity and duration of the insult (e.g. energy). For example, the temperature of the heat source and the duration of its contact with the skin will determine the severity of the burn. The three main burn severity classifications are: superficial partial-thickness (first-degree), deep partial thickness (second-degree), and full thickness (third-degree).

Thermal- mechanical- radiation- laser- and other insult-mediated injury to the skin triggers a “domino effect” and a chain of events of wound progression.

The present invention provides compositions, formulations, compositions and methods for treating, and/or protecting from, a skin disorder, skin energy disruption, skin ailment, skin allergy, skin discomfort, burn, skin discoloration or skin perturbation in a mammalian subject.

In some embodiments of the present invention, improved methods, formulations and compositions are provided for burn therapy.

In some embodiments of the present invention, improved methods, formulations, formulations and compositions are provided for therapy of other and/or chronic skin situations involving disruption of skin barrier.

In some further embodiments of the present invention, improved methods, formulations and compositions are provided for preventing or attenuating blisters, hyperemia, pain, wound progression and subsequent scars after an acute insult to a mammalian's skin.

In some further embodiments of the present invention, improved methods, formulations and compositions are provided for preventing or attenuating blisters, hyperemia, pain, wound progression, inflammation and subsequent scars after a chronic insult to a mammalian's skin.

In some further embodiments of the present invention, improved methods, formulations and compositions are provided for treating, attenuating and/or protecting from, blisters, hyperemia, pain, wound, wound progression and subsequent scars after an acute insult to a mammalian's skin.

In some further embodiments of the present invention, improved methods, formulations and compositions are provided for treating, attenuating and/or protecting from, blisters, hyperemia, pain, wound, wound progression, inflammation and subsequent scars after a chronic insult to a mammalian's skin.

In some additional embodiments of the present invention, improved methods, compositions and compositions are provided for treating, attenuating and/or protecting from, superficial and/or partial deep burns and/or deep burns to a mammalian's skin.

In some embodiments of the present invention, improved methods, formulations and compositions are provided for use as an additional component or the main component in artificial skin and skin complementing compositions and/or formulations.

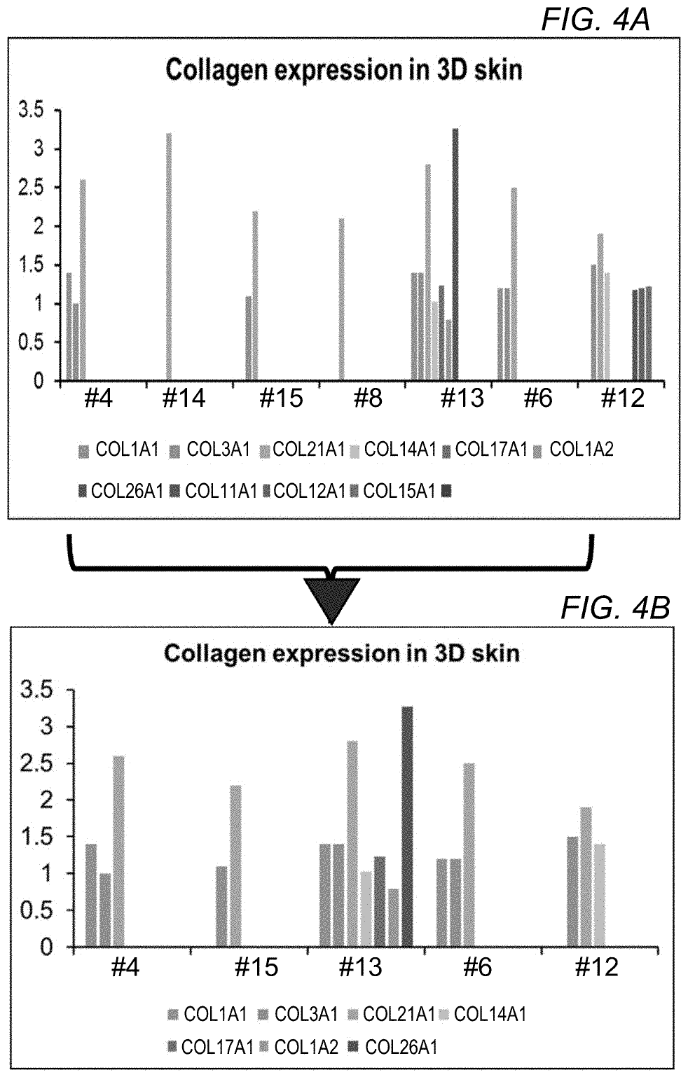

Treatments of UV-exposed skin with new cannabis extracts significantly affected gene expression in the 3D skin, leading to down-regulation of genes and pathways involved in inflammation, immunity and autoimmunity, ECM degradation, and up-regulated genes and pathways involved in collagen synthesis (Table 1; FIG. 4).

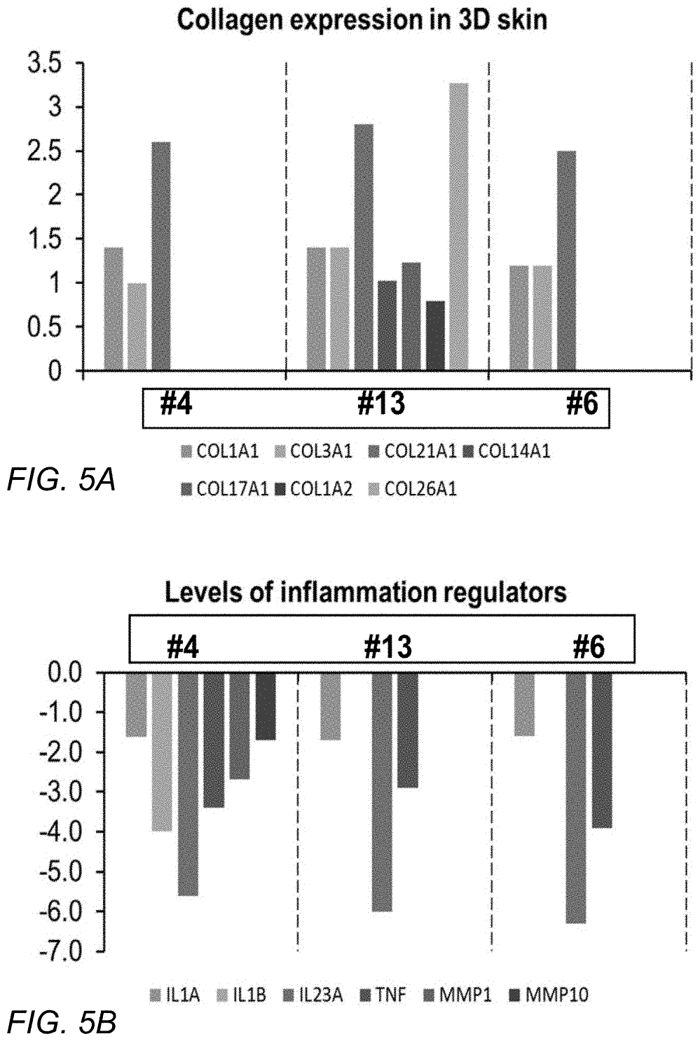

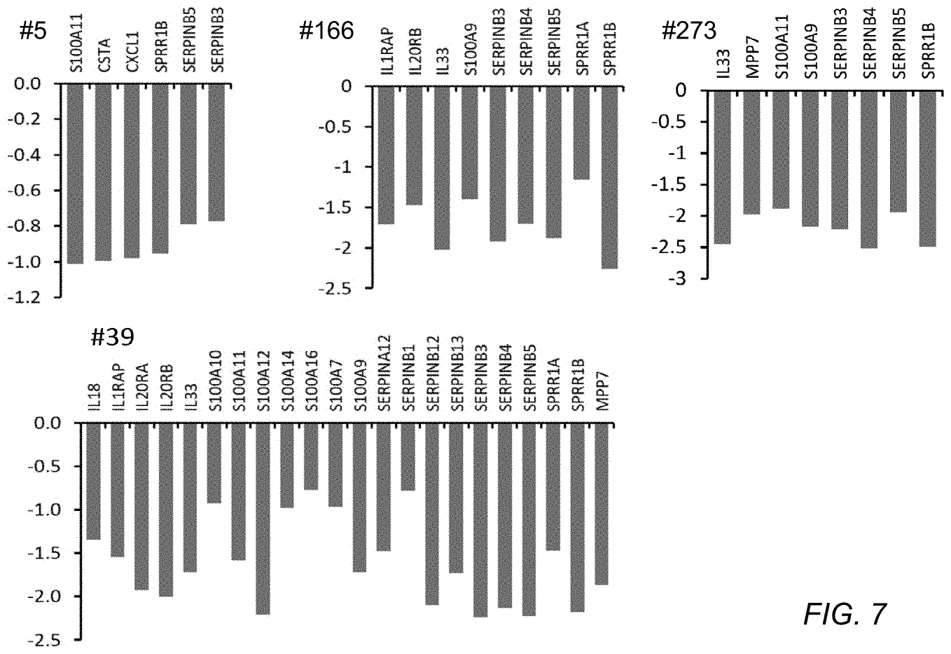

Six out of twenty two extracts profoundly upregulated the levels of collagen genes—COL1A1, COL3A1, COL7A1, COL21A1 and several others (FIG. 4-6). Along with the significant upregulation of collagen, extracts #4, #13 and #6 caused significant down-regulation of MMPs (MMP1, MMP10), interleukins and other pro-inflammatory cytokines, and TNF alpha—one of the key pro-inflammatory molecules and a therapeutic target for the treatment of psoriasis, dermatitis, and a wide array of inflammatory diseases as well as skin inflammation. Extracts #5, #39 and #273 caused profound down-regulation of MMPs, serpins, 5100A and other inflammation and aging-related proteins (FIG. 7). Based on the combined effects on collagen, ECM and inflammation, six lines were the best—#4, #13,#6, # 5, #39, and #273 (FIG. 5-7).

Among those genes down-regulated are members of the Epidermal Differentiation Complex (EDC) genes located on human chromosome 1q21. The EDC comprises 57 genes encoding S100 proteins as well as structural proteins of epidermal cornification. Many of these genes are up-regulated in skin disorders such psoriasis, atopic dermatitis and skin cancers. These genes include members of the Small Proline Rich Region (SPRR), Late Cornified Envelope (LCE), and S100.

One of the extracts (#12) profoundly down-regulated several LCE genes. Four extracts (including #14, #13), down-regulated S100A7 (also known as psoriasin), and β-defensin family member defensin B4A known to be up-regulated in psoriasis. Other genes down-regulated by new extracts and implicated in psoriasis are corneodesmosin (CDSN), CCL20, CXCL8, ELOVL3, members of the serine protease inhibitor (serpin) B genes. Up-regulation of β-defensins is also implicated in basal cell carcinomas. Extract #39 downregulated several S100A genes (S100A7, S100A9, S100A10, S100A11, S100A12, S100A14, S100A16), serpins, and SPRRA1 and A2. S100A7 was also down-regulated by extract #273, along with serpins and other inflammation genes.

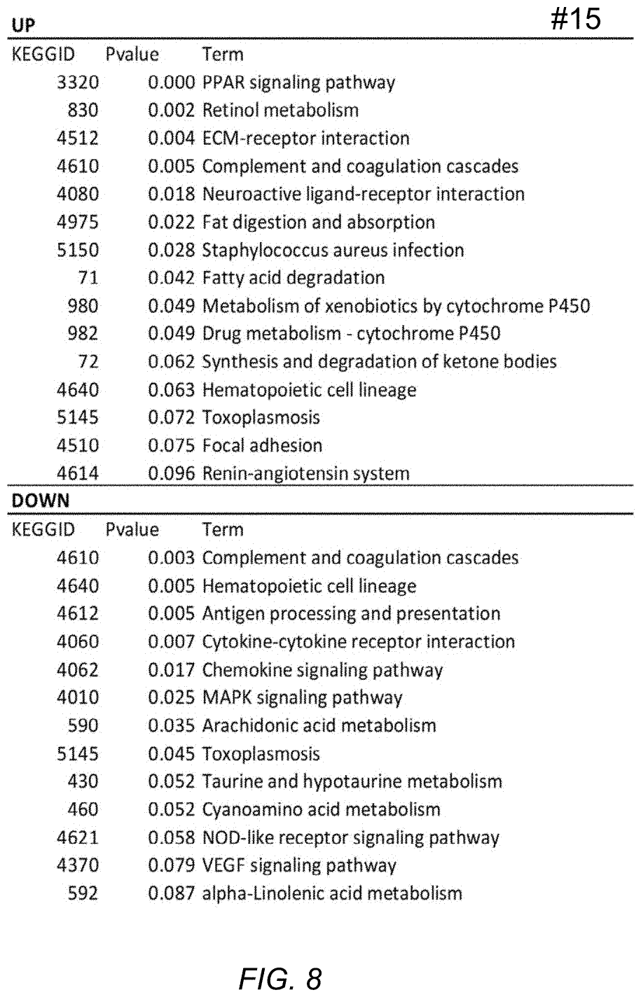

One extracts out of seven (C #15) protected skin tissues from the subsequent exposure to UV, by down-regulating inflammatory pathways and upregulating retinol synthesis pathway (C#15) (FIG. 8). Line#15 also upregulated COL21A1 and down-regulated CXCL8, involved in psoriasis and eczema.

In addition, new extracts may modulate genes and proteins sharing a sequence identity or substantial sequence identity to those genes and proteins listed herein.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

In summary, it has been shown herein that extracts of novel cannabis lines have profound impact on global gene expression in human skin, and positively affect skin responses to UV radiation, and harbor anti-aging, healing and UV-protection properties (FIG. 8). Extracts of new C. sativa lines;

1) Increase collagen and other skin matrix components, as well as:

2) Increase retinol metabolism;

3) Decrease MMP (matrix-degrading enzymes) expression;

4) Profoundly decrease inflammation markers; and

5) Help healing after UV exposure.

Two extracts of seven have UV-protective properties.

Finally, the observed gene expression changes induced by new lines include expression of EDC genes and other genes implicated in non-melanoma skin cancers and inflammatory skin disorders such as psoriasis, which suggests the potential application of new lines and extracts aimed at normalizing the expression of these disease-related genes.

Materials and Methods

Plant Crude Extract Preparation:

Solvent Used: Ethyl Acetate ACS Grade from Fisher cat# E145-4 (99.9% Pure)

Extract Preparation: 3 g of the powdered plant tissue were weighed using an analytical balance. Plant material was placed inside a 250 mL Erlenmeyer flask (clean). 100 mL of Ethyl Acetate was poured into the flask containing the plant material. The flasks were then wrapped with tin foil and shaken continuously (120 rpm) in an incubator @21° C. overnight and in the dark.

After overnight solvent extraction the extracts were filtered through cotton into a 100 mL round bottom flask. The extracts were concentrated to around 2-3 mL using a rotary vacuum evaporator. The extracts were then transferred to a tared 3 dram vial (cat# 60975L Kimble obtained from Fisher Scientific). The leftover solvent was evaporated to dryness in an oven overnight @50° C. to eliminate the solvent completely. Mass of each extract was recoded.

Bioassay Preparation

Preparation of 60 mg/mL Stock Solutions.

The stocks were prepared weighing a 3-6 mg of crude extract into a micro centrifuge tube. The crude extract was dissolved in DMSO (Dimethyl sulfoxide anhydrous from Life technologies cat # D12345) to reach 60 mg/mL final concentration and stored at −20° C.

Preparation of Crude Extracts for Bioassay.

Appropriate cell culture media (in these experiments RPMI+10% FBS or EMEM+10% FBS) was used to dilute the 60 mg/mL stock. The stocks are allowed to thaw then added to the cell culture media, mixed thoroughly to ensure they are in solution and filtered through a 0.22 um syringe filter. These filtrates were ready to be applied to cells and tested.

Methods:

EpiDerm full thickness 400 (EFT-400) Skin model (Mat Tek) was used.

EpiDermFT has normal skin tissue structure with differentiated dermis and epidermis, and consists of normal, human epidermal keratinocytes (NHEK) and normal, human dermal fibroblasts (NHFB) cultured to form a multilayered model of the human dermis and epidermis. It exhibits in vivo-like growth and morphological characteristics whereby cells sustain differentiation and metabolic status similar to those of human epidermis. The model is widely used and accepted for platform for studying toxicity of cosmetics and topical agents, effects of ionizing radiation, skin carcinogenesis and wound healing. Tissues were equilibrated in EFT-400 for 24 h (overnight) then culture media EFT-400 was replaced and incubated for another 24 h.

Exposure:

Tissues were exposed for 2 min to UVC, in a biosafety cabinet. Distance from the light source was set to 10 cm; only 3 wells of the 6 well plate were exposed at a time (to make the distance effective in all 3 wells).

The experiment 1 was divided into 2 groups:

A=UV protection experiment, this means 15 uL of crude extract solution or control were applied to the tissue before exposure.

B=UV healing experiment, this means 15 uL of crude extract solution or control were applied to the tissue after exposure.

Control was divided into 3 as to keep all variations with a control: Ct (PBS),

Ct+PBS then UV, Ct +UV then PBS, the same approach was done for DMSO (0.017% final concentration) samples: DMSO on top, DMSO then UV, UV then DMSO.

Application volume of extracts: 15 uL of these solutions were applied on top of the tissues (inside the cup holding the tissue) ensuring even coverage of the tissue surface.

Tissues once treated were allowed to equilibrate at 37C in an incubator with 6% CO2, for 48 hours. Then all tissues were frozen using liquid N2 and stored at −80C.

Experiment 2 was divided into two groups and used extracts #5, #31, #39, #49, #69, #81, #11, #166, #267 and #273 as well as #10, #11, #155, #156 and #207.

Extracts were dissolved in coconut oil, 0.01 ug/uL, 15 μL was applied on top of the tissues for 24 hours. Vehicle—coconut oil, served as control. The samples were harvested in 24 hours after the treatment.

Gene Expression Profiling:

Three tissues per group were used for the analysis of gene expression profiles. RNA was extracted from tissues using TRIzol® Reagent (Invitrogen, Carlsbad, Calif.), further purified using an RNAesy kit (Qiagen), and quantified using Nanodrop2000c (ThermoScientific). Afterwards, RNA integrity and concentration were established using 2100 BioAnalyzer (Agilent). Sequencing libraries were prepared using Illumina's TruSeq RNA library preparation kits, and global gene expression profiles were determined using the Next 500 Illumina deep-sequencing platform at the University of Lethbridge Facility. Statistical comparisons between the control and treatment groups were performed using the DESeq Bioconductor package (version 1.8.3) and the baySeq Bioconductor package (version 1.10.0). Clustering of the samples was assessed with multidimensional scaling (MDS) plots built using the plotMDS function from the edgeR Bioconductor package. Features with a false discovery rate (FDR)<0.1 (10% false positive rate) were considered differentially expressed between conditions.

The functional annotations of differentially expressed genes were performed using David, GO (Gene Ontology) Elite, and GO-TermFinder. Pathways were visualized using Pathview/KEGG and DAVID bioinformatics platforms DAVID Bioinformatics Resources 6.7 KEGG Pathway platforms.

Result Figures

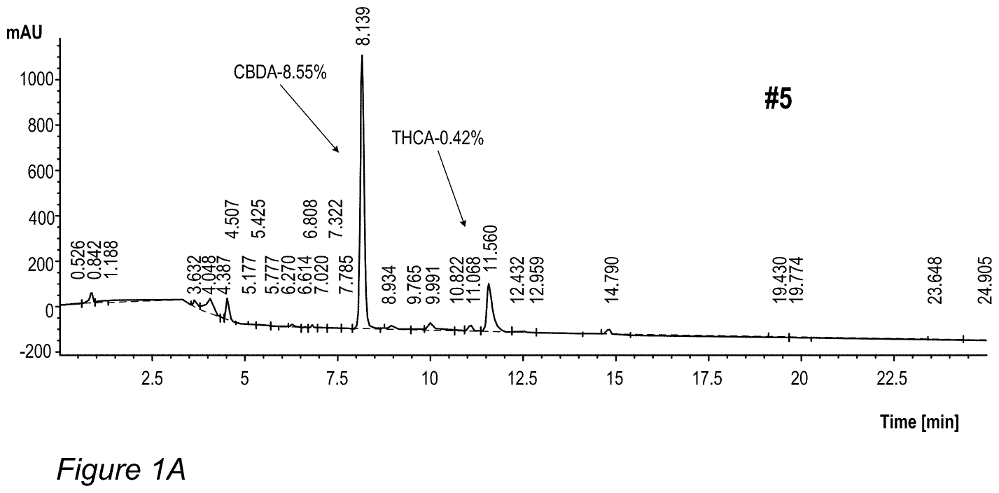

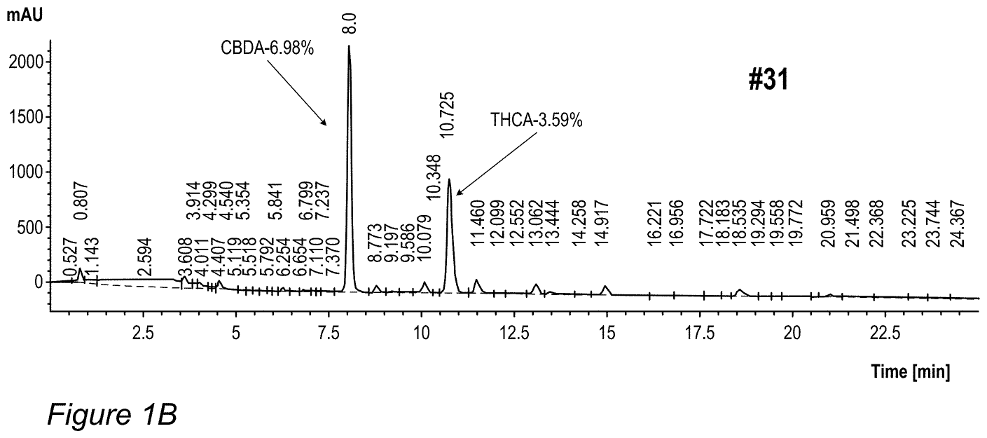

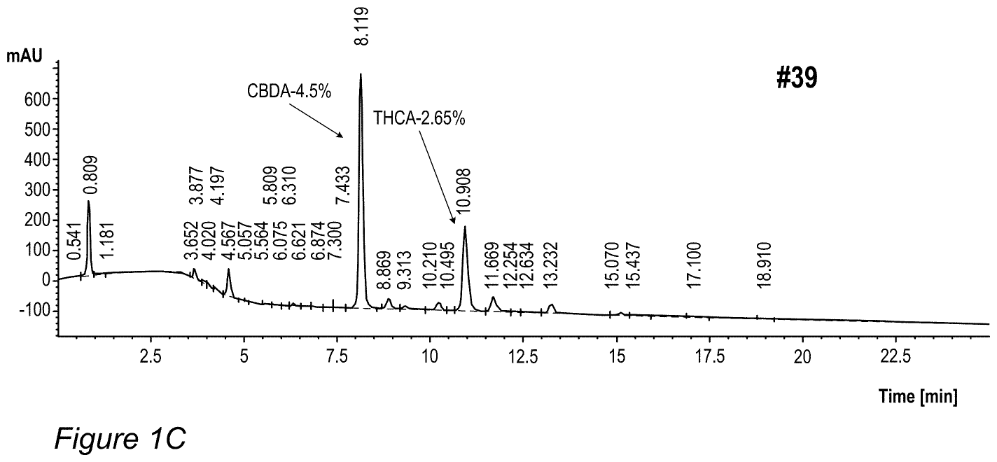

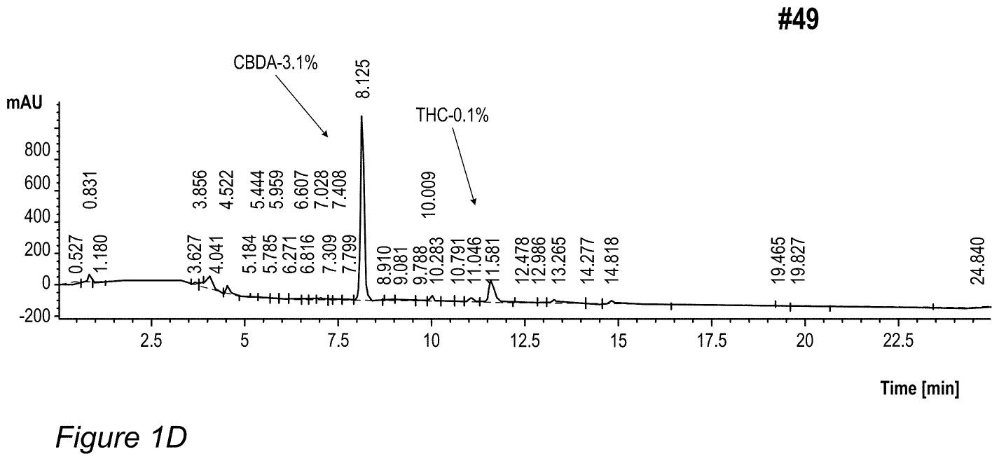

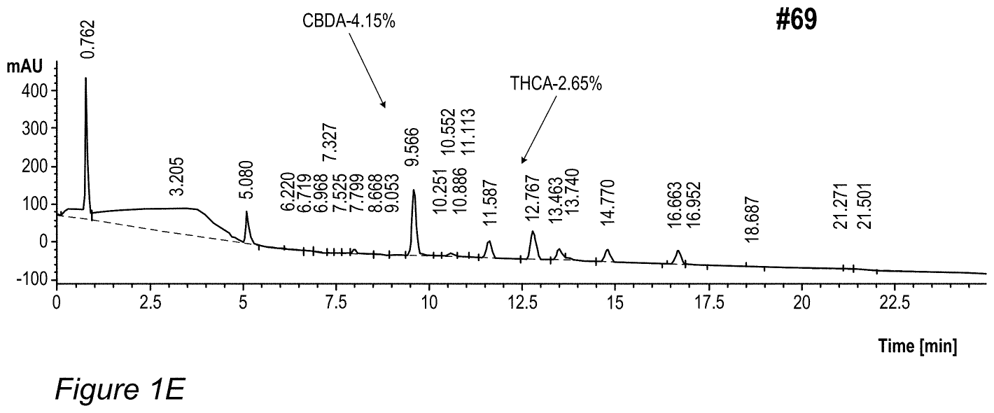

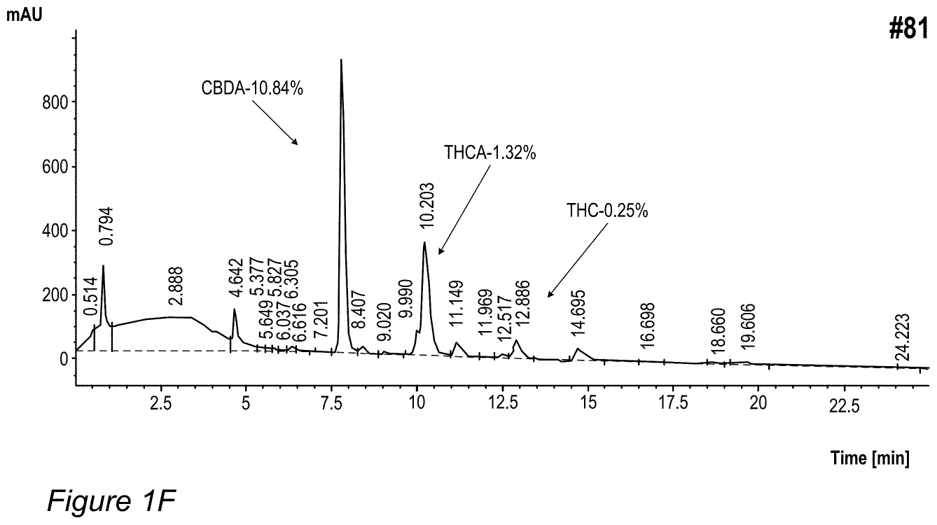

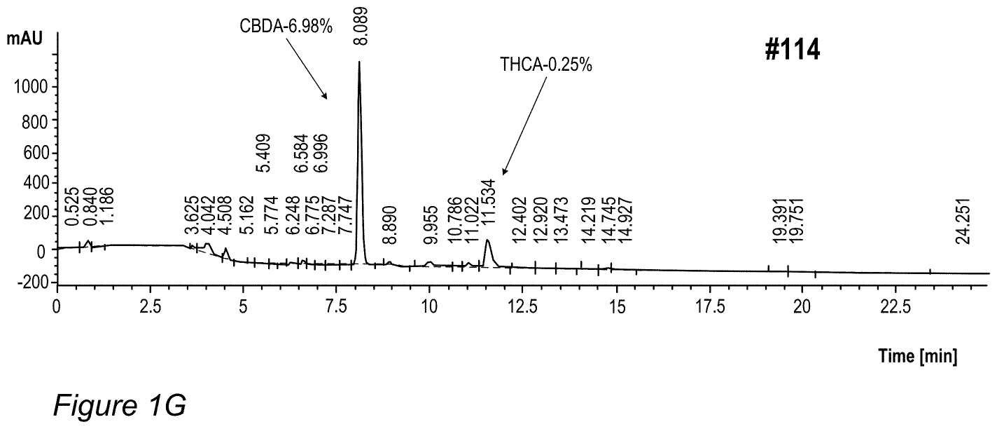

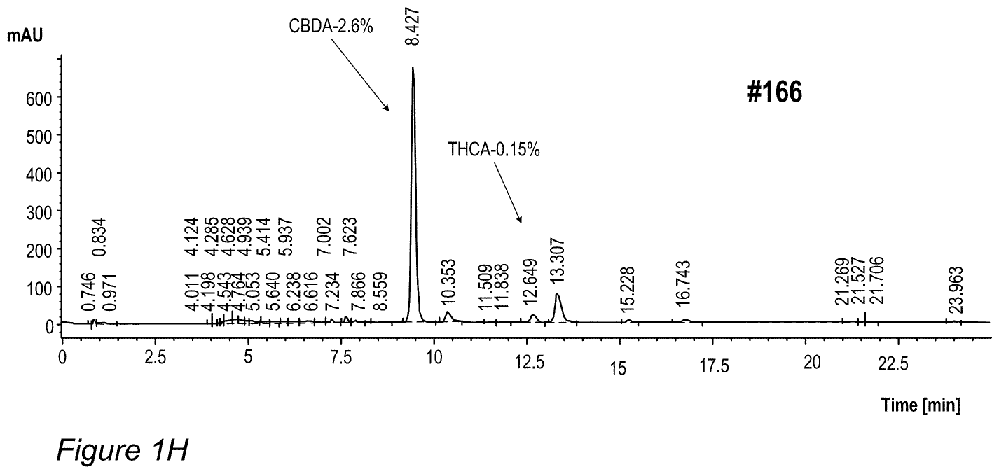

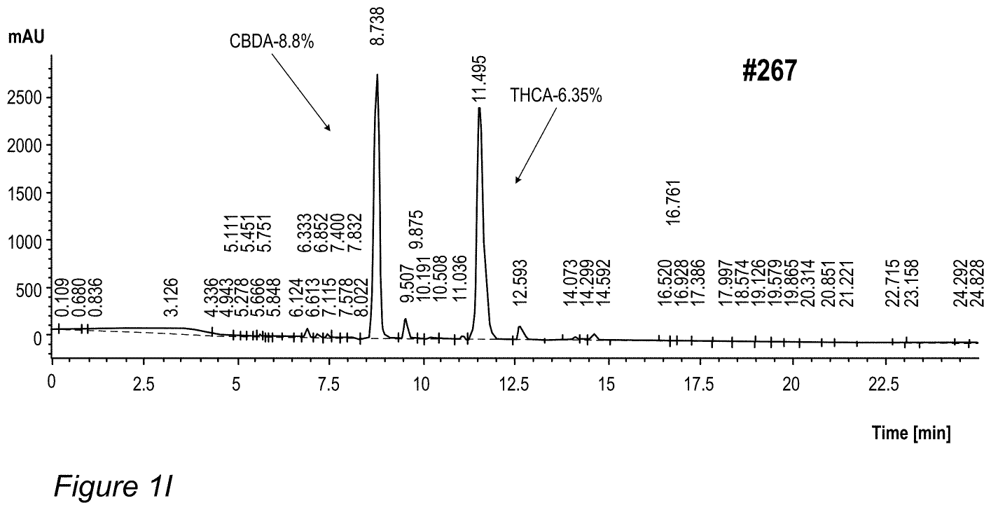

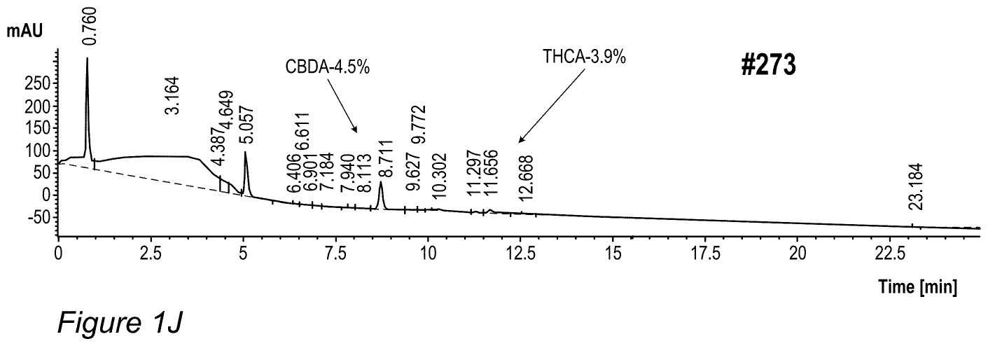

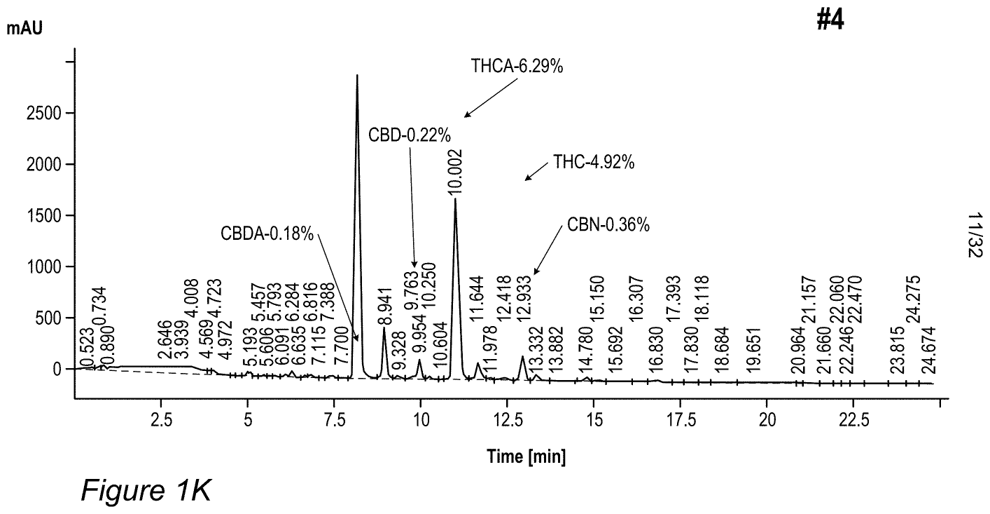

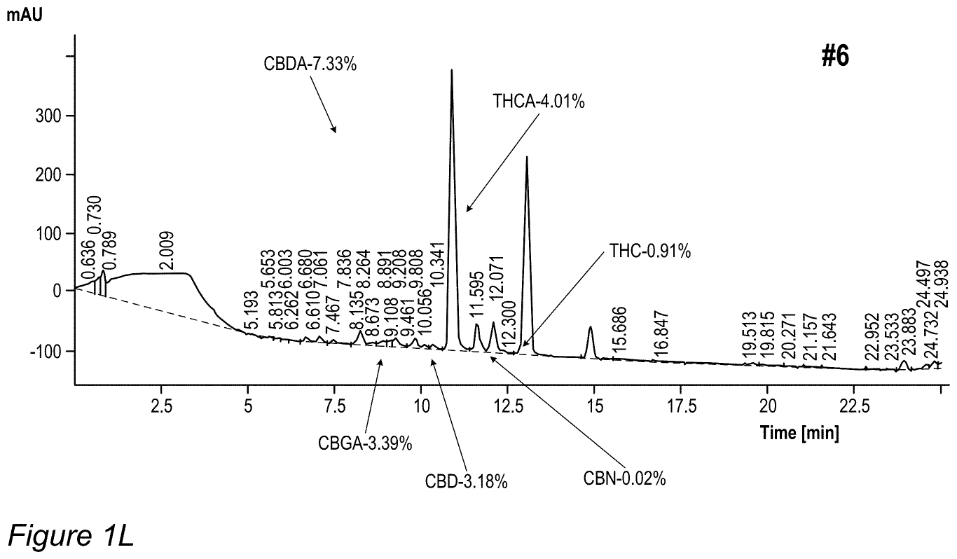

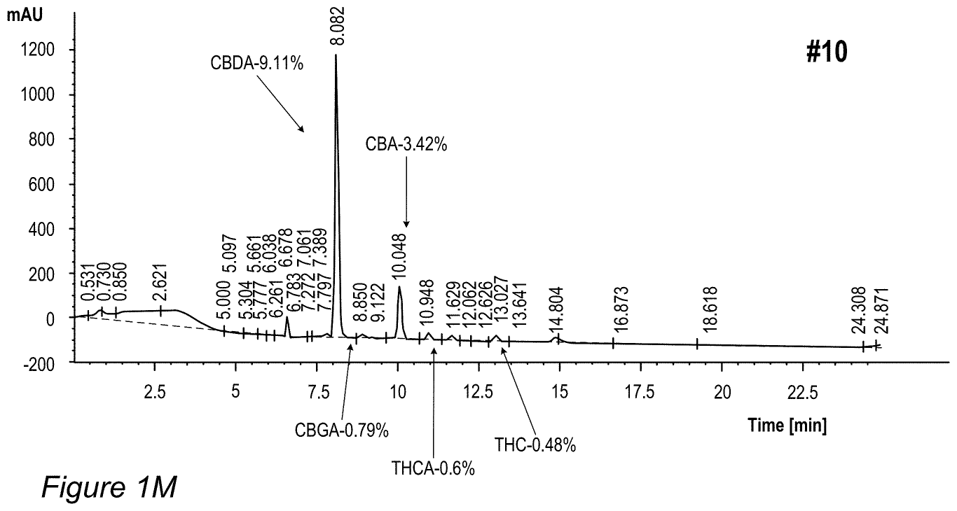

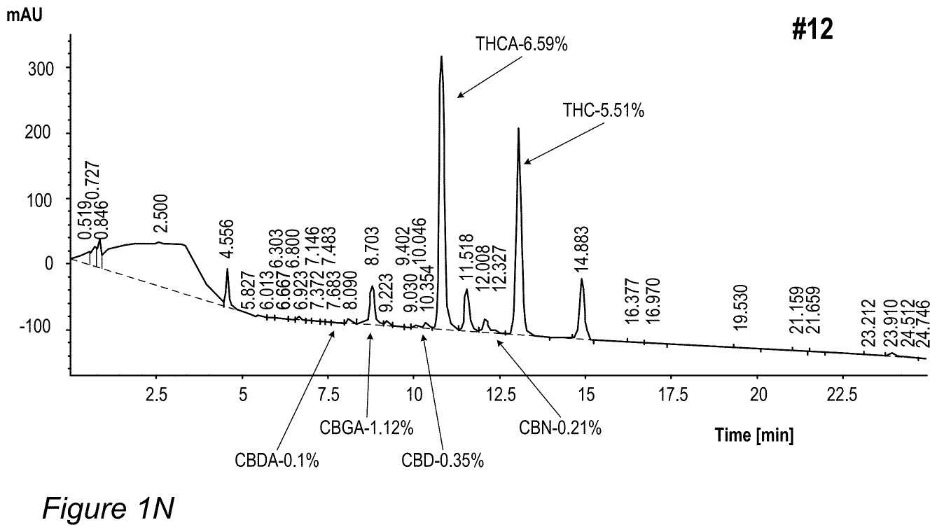

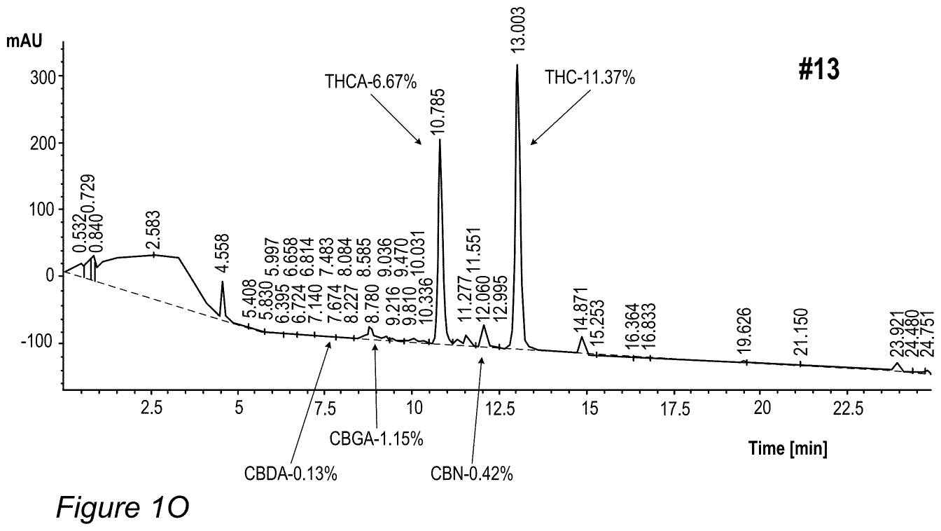

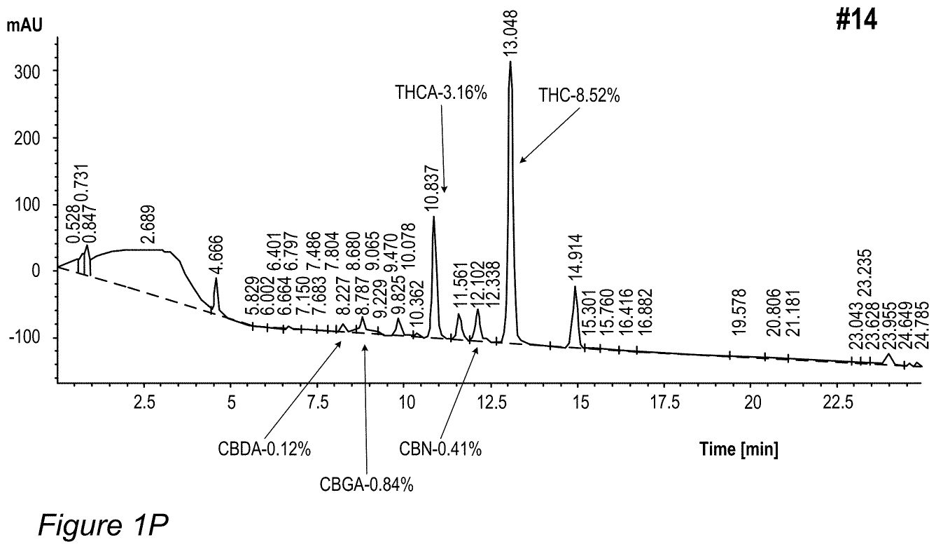

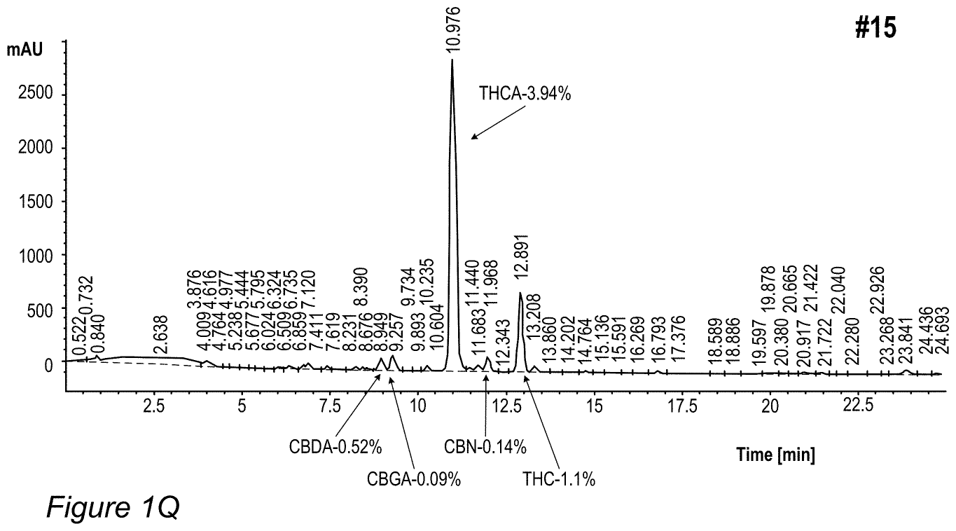

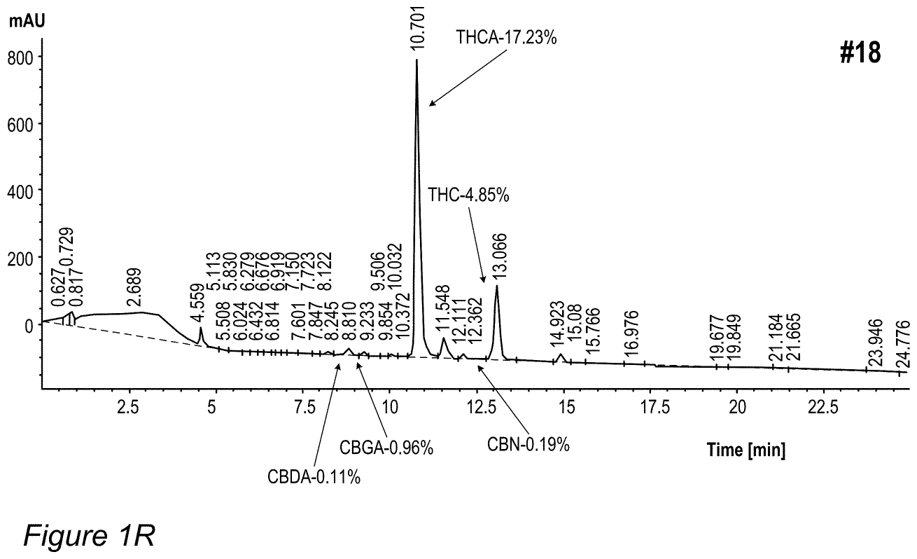

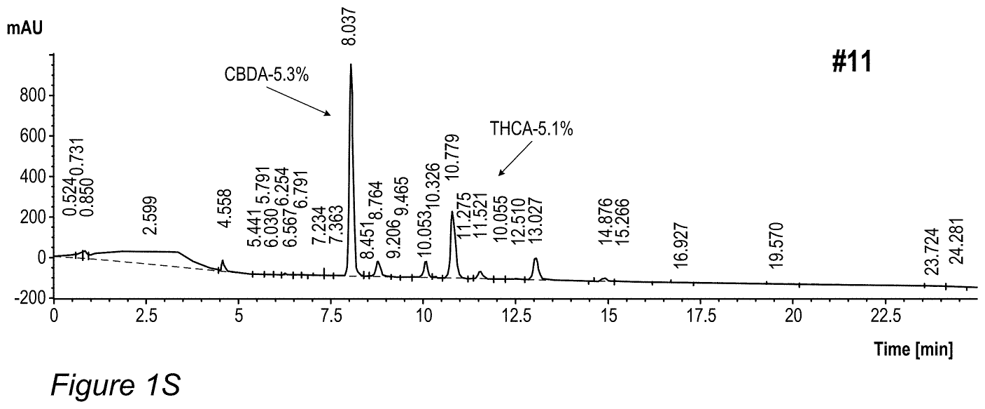

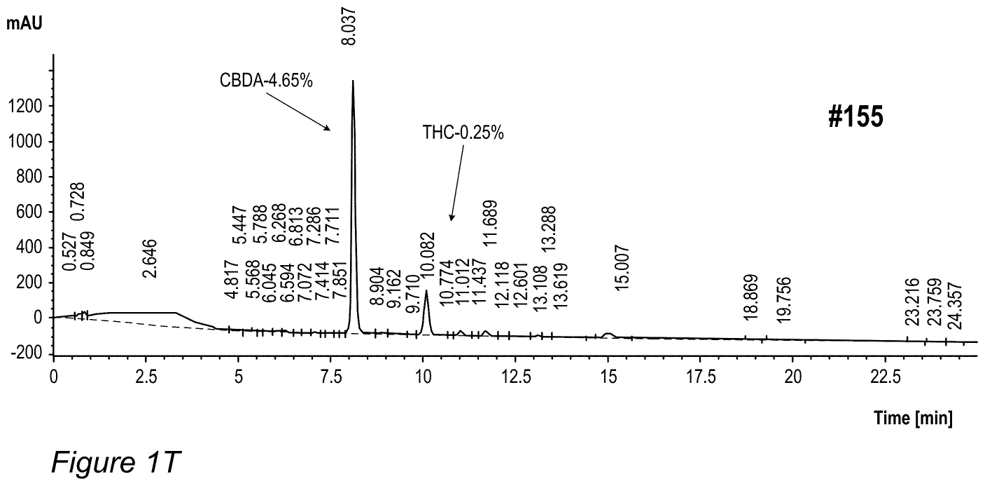

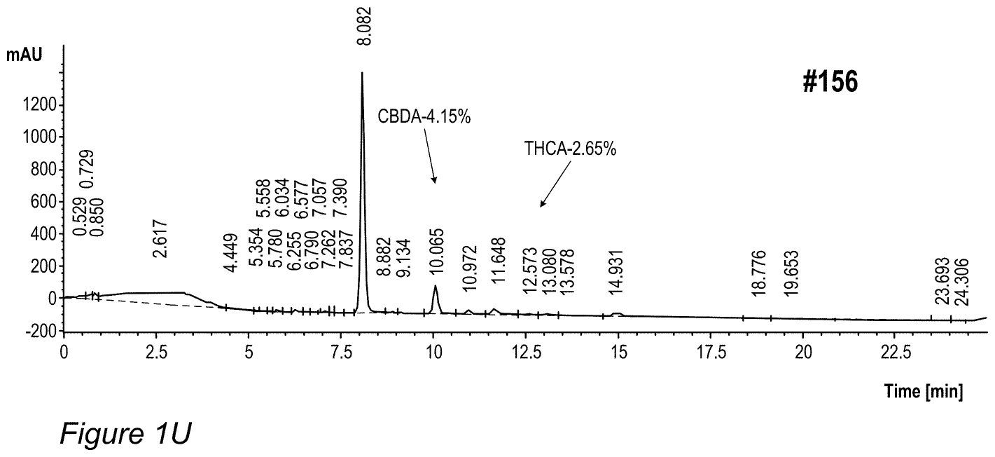

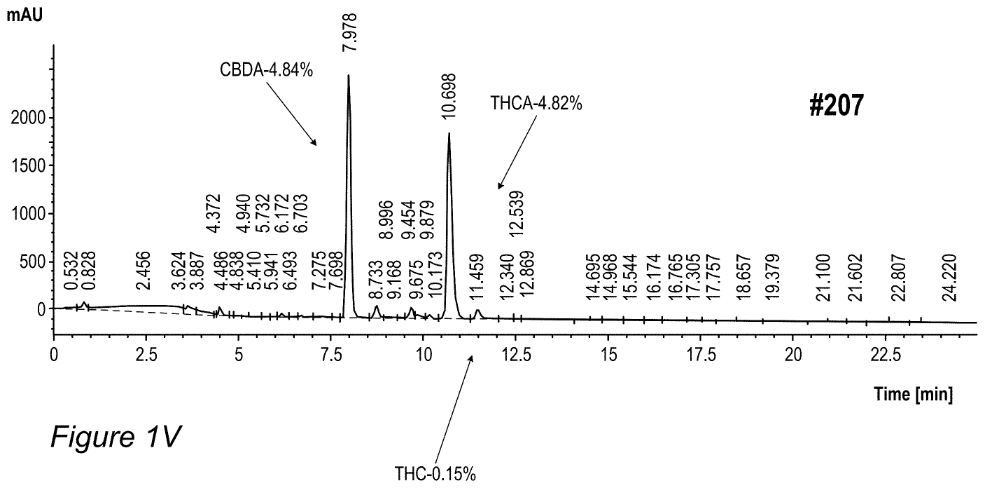

FIG. 1 High performance liquid chromatography (HPLC)
profiles of tested lines, in accordance with some
embodiments of the present invention.

| FIG. 1 panel | LINES (#) | THC | CBD |
|---|---|---|---|
| FIG. 1A | 5 | 0.27 | 8.46 |
| FIG. 1B | 31 | 3.54 | 6.90 |
| FIG. 1C | 39 | 2.6 | 4.4 |
| FIG. 1D | 49 | 0.1 | 3.05 |
| FIG. 1E | 69 | 2.56 | 4.1 |
| FIG. 1F | 81 | 1.37 | 10.38 |
| FIG. 1G | 114 | 0.18 | 6.92 |
| FIG. 1H | 166 | 0.1 | 2.5 |
| FIG. 1I | 267 | 6.8 | 8.2 |
| FIG. 1J | 273 | 3.8 | 4.4 |
| FIG. 1K | 4 | 10.44 | 0.38 |
| FIG. 1L | 6 | 4.43 | 9.61 |
| FIG. 1M | 8 | 14.72 | 0.41 |
| FIG. 1N | 12 | 12.29 | 0.44 |
| FIG. 1O | 13 | 17.22 | 0.21 |
| FIG. 1P | 14 | 11.3 | 0.42 |
| FIG. 1Q | 15 | 4.57 | 0.47 |
| FIG. 1R | 18 | 19.96 | 0.1 |
| FIG. 1S | 11 | 5.2 | 5.0 |
| FIG. 1T | 155 | 0.22 | 4.59 |
| FIG. 1U | 156 | 0.26 | 5.0 |
| FIG. 1V | 207 | 4.7 | 4.7 |

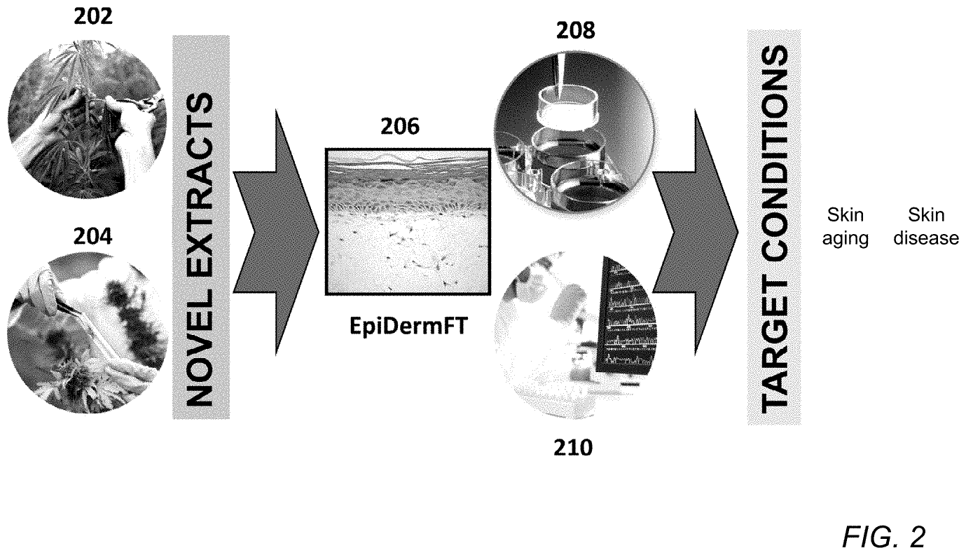

FIG. 2. shows a simplified schematic, part-pictorial illustration of a method for identifying new C. sativa lines with anti-aging properties, in accordance with some embodiments of the present invention. Novel extracts were applied to the EpiDermFT tissues, and their effects on global gene expression were analyzed.

Step 202—a cultivar growing step. Around 250 unique marijuana and around 120 unique hemp cultivars were used to generate approximately 1,200 marijuana/marijuana, hemp/hemp and hemp/marijuana hybrids. Cultivars are typically grown in soil/vermiculite (2:1) mix. First, plants are grown under 16 h day, 8 h night for approximately 6 weeks when they were moved to another grow room and grown at 12 h day and 12 h night for another 6-8 weeks until they developed mature flowers. In both rooms, they were grown under the high pressure sodium (HPS) lights of ˜400 W/m2. Collected flowers were then tested for cannabinoids and terpenoids and those with most diversity in composition, or those that had highest amount of one or more cannabinoid or terpenoid or those that had the presence of unique terpenoids were used for breeding. The progeny of these crosses was then grown and further tested for cannabinoids/terpenoids as well as for growth parameters, such as height, response to nutrients, responses to pathogens, amongst others.

In some cases, these plants were then crossed again using siblings with similar traits (cannabinoids/terpenoids for example). The seeds of these cultivars (resulting from crosses) are stored at +4° C. in the fridge in the locked cage. Approximately 600 strains with the best parameters, such as diversity of cannabinoids and terpenoids, plant growth vigor (germination rate, mutation time, yield of flowers, nutrients response, response to pathogens, size of flowers) and other features such as distinct smell for example were germinated and approximately 400 extracts were made. Most organic solvents can be used for the extraction. In one experiment ethyl acetate was used. This should not be deemed as limiting. For extract preparation, 3 g of the powdered flower tissue were used in 100 ml of ethyl acetate in a 250 mL Erlenmeyer flask. The flasks were then wrapped with tin foil and shaken continuously (120 rpm) in an incubator at 21° C. overnight and in the dark. After overnight solvent extraction the extracts were filtered through cotton into a 100 ml round bottom flask. The extracts were concentrated to around 2-3 ml using a rotary vacuum evaporator. The extracts were then transferred to a tared 3 dram vial. The leftover solvent was evaporated to dryness in an oven overnight at 50° C. to eliminate the solvent completely. Mass of each extract was recorded, and the extracts were stored at −20° C. The stocks were prepared weighing a 3-6 mg of crude extract into a micro centrifuge tube. The crude extract was dissolved in DMSO (Dimethyl sulfoxide anhydrous) to reach 60 mg/mL final concentration and stored at −20° C. Around 400 solvent-based crude extracts of flowers were thus generated.

In a tissue preparation step 206—3D EpiDermFt tissues of normal skin epithelial tissues were used.

In an extract biological assay step 208, many of the selected extracts were tested as follows. Appropriate cell culture media (for example RPMI+10% FBS or EMEM+10% FBS) was used to dilute the 60 mg/mL stock. Appropriate amounts of stock extract were added to the media used for 3D tissues, mixed thoroughly to ensure they are in solution and filtered through a 0.22 um syringe filter. These filtrates were ready to be applied to 3D tissues and tested. For example, to achieve the concentration of 0.007 mg/ml, 2.45 μl of stock extract (60 mg/ml) was added to 21 ml of medium.

In a bioinformatics analysis step 210, gene expression data were obtained from harvested tissue and altered pathways were analyzed bioinformatically.

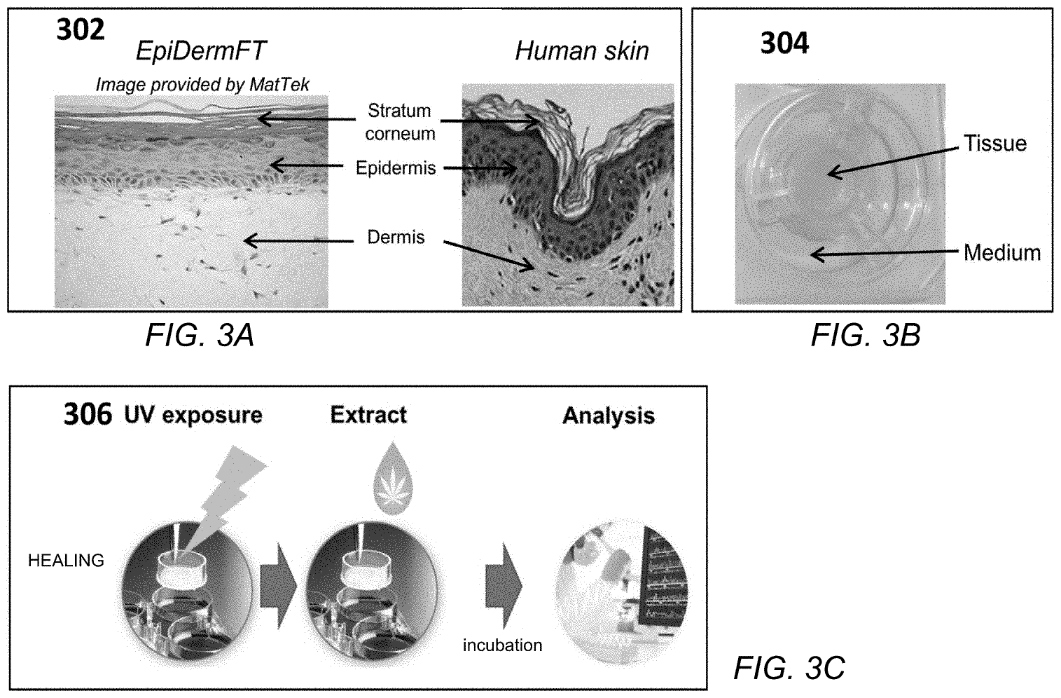

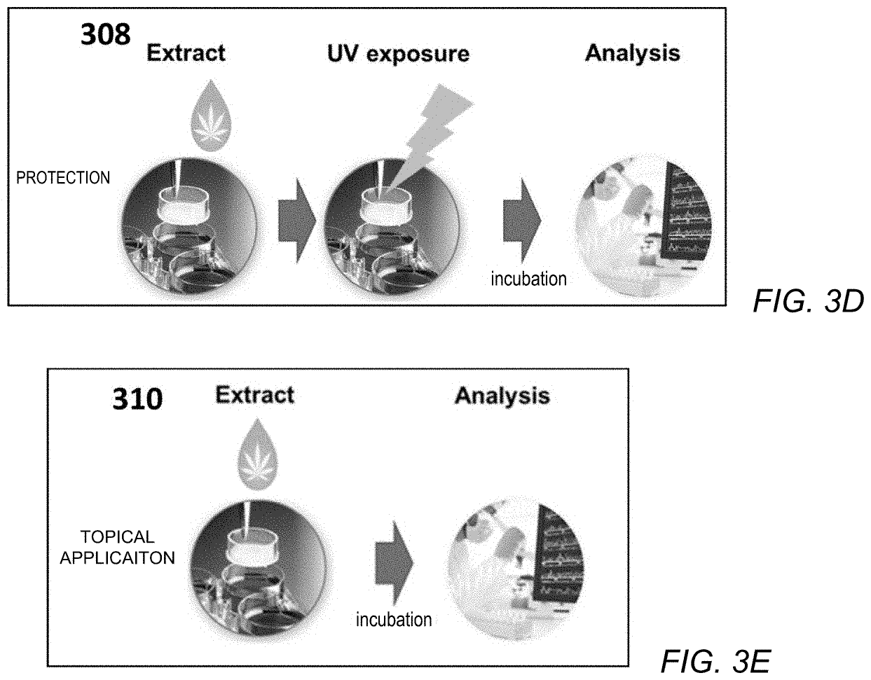

FIGS. 3A-3E. EpiDermFt tissues and experimental set-up. FIG. 3A. EpiDermFT (Mattek) has normal skin tissue structure with differentiated dermis and epidermis and is constructed from human-derived epidermal keratinocytes and dermal fibroblasts. It exhibits in vivo-like growth and morphological characteristics whereby cells sustain differentiation and metabolic status similar to those of human epidermis. The model is widely used and accepted for platform for studying toxicity of cosmetics and topical agents, effects of ionizing radiation, skin carcinogenesis and wound healing. FIG. 3B. Tissue insert in a well with medium. FIG. 3C. Scheme of healing experiments. For the healing experiment, tissues were exposed to UV, then treated with extracts and used for global gene expression profiling. FIG. 3D. For protection experiments, tissues were treated with extracts and then exposed to UV, and further used for gene expression profiling. FIG. 3E. To analyze the direct anti-aging effects of extracts on skin, extracts were dissolved in coconut oil and applied directly to the skin model surface.

In a tissue preparation step 302—3D EpiDermFt tissues of normal skin epithelial tissues were used.

Further in an incubation step 304, 3D tissues are inserted in a well with medium. Tissues were equilibrated in EFT-400 for 24 h (overnight) then culture media EFT-400 was replaced and incubated for another 24 h.

In UV exposure step 306, tissues were exposed for 2 min to UVC, in a biosafety cabinet. Distance from the light source was set to 10 cm. In extract treatment step, all crude extracts were diluted from a 60 mg/mL stock (the stock is prepared in DMSO). For this experiment, a final concentration of 0.01 ug/uL in 30% glycerol-PBS was used. 24 h after UV exposure, 15 uL of 0.01 ug/uL extract solution or control (PBS alone) were applied to the tissue after exposure. Control samples consisted of the following samples: PBS-exposed only, PBS and DMSO added after UVC exposure (crude extracts are stored in DMSO). In the analysis step, the samples were harvested in 24 h after application of extracts and used for the analysis of mRNA by sequencing. Bioinformatics analysis of mRNA revealed changes in biological pathways associated with skin healing.

In UV protection step 308, tissues were first treated with extracts for 24 h (as per step 306) and then exposed for 2 min to UVC, in a biosafety cabinet and tissue harvested for the analysis in 24 h. Bioinformatics analysis of mRNA revealed changes in biological pathways associated with skin protection.

In topical application step 310, to analyze the direct anti-aging effects of extracts on skin, extracts were dissolved in coconut oil and applied directly to the skin model surface for 24 h upon which the samples were taken for the analysis. Bioinformatics analysis of mRNA revealed changes in biological pathways associated with skin anti-aging properties.

FIG. 4A shows the effects of new cannabis lines on collagen gene expression—experiment 1. Genes with a False Discovery Rate (FDR)-adjusted p-value<0.05 and log2 fold change>0.6 (1.5× change) were considered differentially expressed. Levels of gene expression are shown as relative expression units, fold change are compared to control. Upper panel shows results of new extracts #4, 14, 15, 8, 13, 6, 12, whereas (FIG. 4B)—the lower panel focuses on the lines that caused changes in the two or more collagen genes.

FIG. 5A shows the effects of three best new cannabis lines on expression of genes involved in collagen, inflammation and extracellular matrix (ECM) maintenance—experiment 1. These lines affected expression of 3 or more collagen genes. FIG. 5B shows the effects of three best new cannabis lines on expression of genes involved in inflammation. Genes with a False Discovery Rate (FDR)-adjusted p-value<0.05 and log2 fold change>0.6 (1.5× change) were considered differentially expressed. Levels of gene expression are shown as relative expression units, fold change are compared to control.

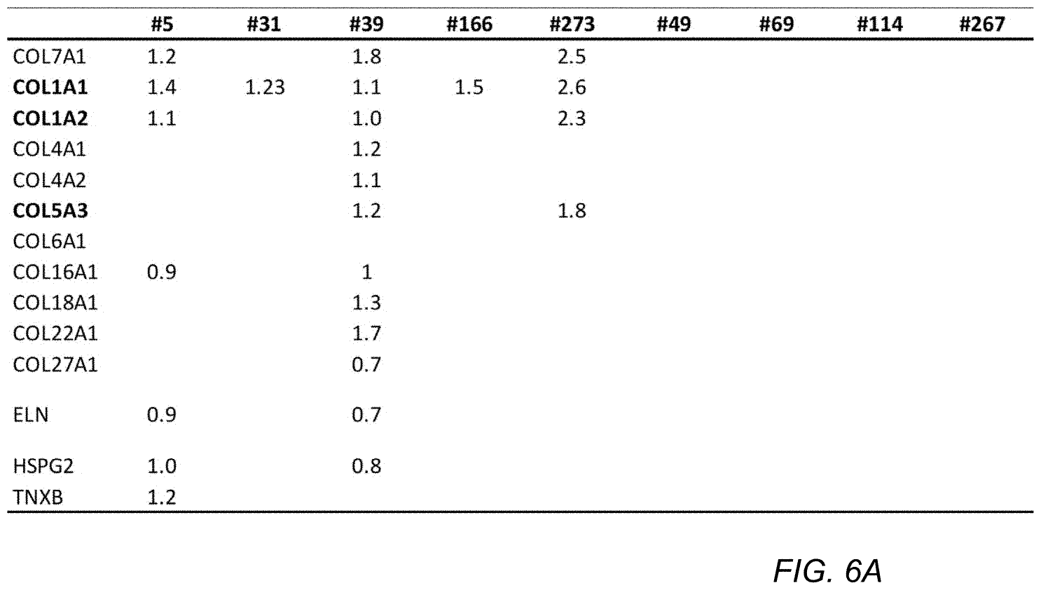

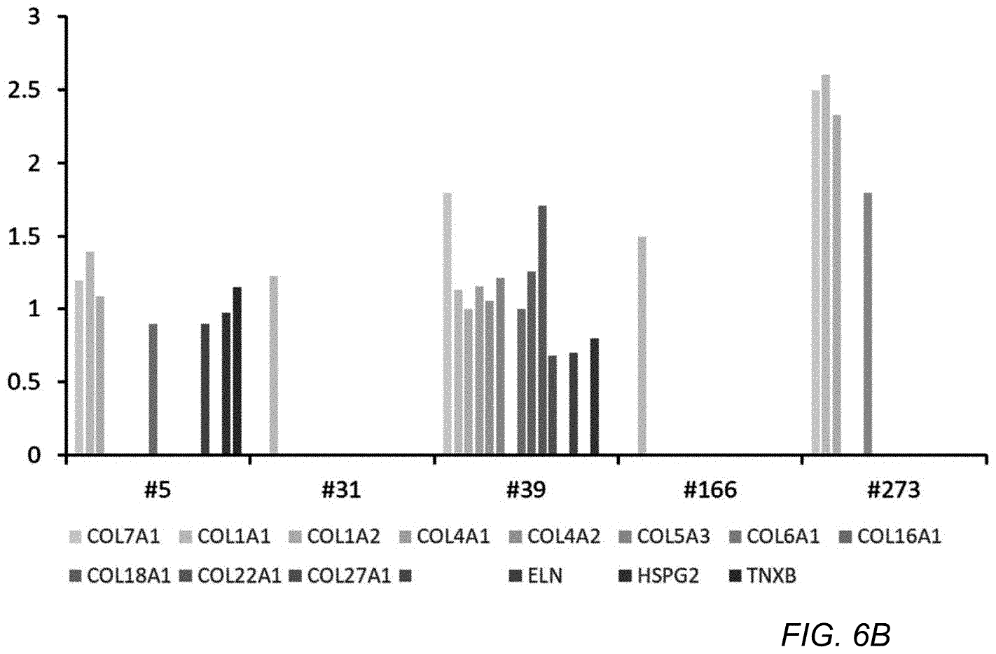

FIG. 6A shows the effects of new cannabis lines on collagen gene expression—experiment 2. Extracts were dissolved in coconut oil and applied to the surface of the skin tissues. Genes with a False Discovery Rate (FDR)-adjusted p-value<0.10 and log2 fold change>0.6 (1.5× change) were considered differentially expressed. Levels of gene expression are shown as relative expression units, fold change are compared to oil-treated control. FIG. 6A in a table form shows results of all extracts # 3, 31, 39, 49, 69, 114, 166, 267, 273, whereas FIG. 6B focuses on the lines that caused changes in the two or more collagen genes, and affected elastin, tenascin and heparan sulfate proteoglycan 2 (perlecan) genes.

FIG. 7 shows the effects of new cannabis lines on expression of genes involved in skin inflammation and disease—S100A, serpins, SPRR, IL and other genes—experiment 2. Extracts were dissolved in coconut oil and applied to the surface of the skin tissues. Genes with a False Discovery Rate (FDR)-adjusted p-value<0.10 and log2 fold change>0.6 (1.5× change) were considered differentially expressed. Levels of gene expression are shown as relative expression units, fold change are compared to oil-treated control.

FIG. 8. Mechanisms affected by UV exposure is the UV-protection experiment by line #15.

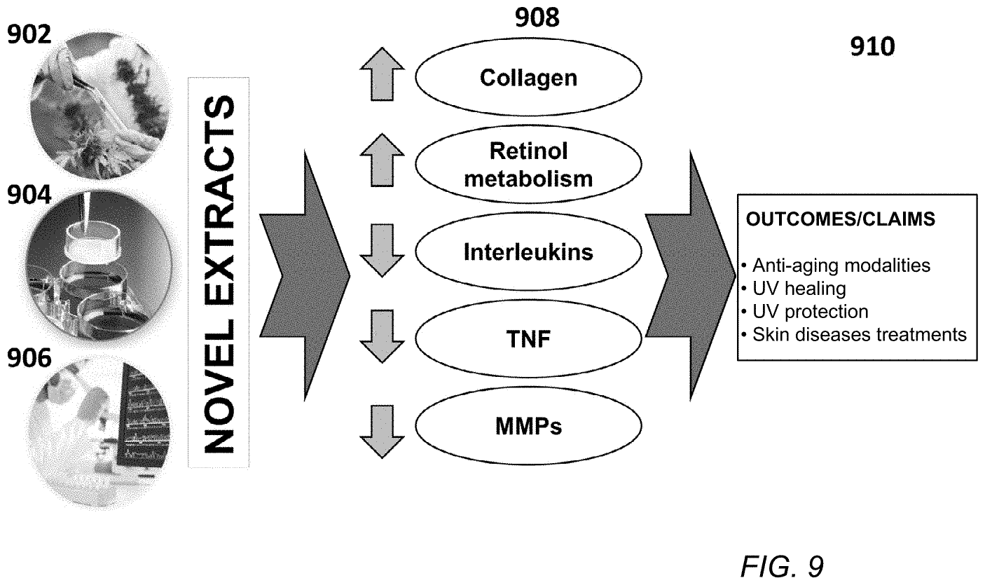

FIG. 9. Schematic summary of the main results.

In a step for developing hybrids with novel skin healing components 902, protective and anti-aging extracts hybrids of different cannabis varieties are created and full flower extracts of various hybrids were prepared.

In a UV application step 904, skin epithelial 3D tissues were either pre-treated with extracts followed by UV, treated with UV followed by extracts or just treated with extracts.

In a detecting down-regulated pathways step 906, the data obtained from mRNA-seq were used to identify downregulated pro-inflammatory pathways.

In a bioactive compounds detection step 908, extracts upregulating collagen production and retinol metabolism and downregulating TNF, interleukins or MMPs are identified.

In a extracting compounds step 910, such extracts are used for generation of novel treatments for skin care.

The present invention further provides new unique cannabis lines, cultivars, hybrids, extracts, dried powders from the extracts, compositions comprising the powders or parts thereof, compounds derived therefrom, pharmaceutical compositions comprising the compound(s) and methods for their use in skin treatment and modalities. The method includes generation of unique lines, whole plant extract preparation, treating skin cells and/or proliferative skin cells with extracts in amount sufficient to kill proliferative/cancer cells while sparing normal (non-proliferative) ones. The modulation of cell proliferation, growth and death results in efficient elimination of cancer cells in response to the anti-cancer therapies and modalities of the present invention.

The present invention further provides new unique cannabis lines, cultivars, hybrids, extracts, dried powders from the extracts, compositions comprising the powders or parts thereof, compounds derived therefrom, pharmaceutical compositions comprising the compound(s) and methods for their use in skin diseases and disorder treatment.

The present invention provides novel cannabis lines which are able to down-regulate a plurality of biological pathways. These pathways maybe metabolic pathways and/or signaling pathways. For example, line #4 downregulates the pathways appearing in the table hereinbelow.

Dosage Forms

The compositions of the present invention may be provided in any suitable dosage form. According to some embodiments, the dosage form is an oral dosage form. Oral dosage forms comprise liquids (solutions, suspensions, and emulsions), semi-solids (pastes), and solids (tablets, capsules, powders, granules, premixes, and medicated blocks).

Some examples of oral dosage forms in the art include, WO90/04391, which discloses an oral dosage form of omega-3 polyunsaturated acids to overcome the problems of diseases. It is known to supply said acids in soft gelatine capsule shells.

EP 2 240 581 B1 discloses a gelatine capsule for pharmaceutical use with a controlled release of active ingredients and a process for the preparation of said gelatin capsules. During said process xylose is added to the liquid gelatin from which afterwards gelatin capsules are formed. Gelatin capsules manufactured according to the process provide retarded release of active ingredients.

U.S. Pat. No. 7,264,824 discloses and oral dosage form for food and food supplements, as well as dietetics comprising polyunsaturated acids in a xylose-hardened gelatine capsule with a retarded release time.

According to some embodiments of the present invention, the compositions described herein may be in a suspension or emulsion.

A suspension is a coarse dispersion of insoluble drug particles, generally with a diameter exceeding 1 μm, in a liquid (usually aqueous) medium. Suspensions are useful for administering insoluble or poorly soluble drugs/components or in situations when the presence of a finely divided form of the material in the GI tract is required. The taste of most drugs is less noticeable in suspension than in solution, due to the drug being less soluble in suspension. Particle size is an important determinant of the dissolution rate and bioavailability of drugs in suspension. In addition to the excipients described above for solutions, suspensions include surfactants and thickening agents. Surfactants wet the solid particles, thereby ensuring the particles disperse readily throughout the liquid. Thickening agents reduce the rate at which particles settle to the bottom of the container. Some settling is acceptable, provided the sediment can be readily dispersed when the container is shaken. Because hard masses of sediment do not satisfy this criterion, caking of suspensions is not acceptable.

An emulsion is a system consisting of 2 immiscible liquid phases, one of which is dispersed throughout the other in the form of fine droplets; droplet diameter generally ranges from 0.1-100 μm. The 2 phases of an emulsion are known as the dispersed phase and the continuous phase. Emulsions are inherently unstable and are stabilized through the use of an emulsifying agent, which prevents coalescence of the dispersed droplets. Creaming, as occurs with milk, also occurs with pharmaceutical emulsions. However, it is not a serious problem because a uniform dispersion returns upon shaking. Creaming is, nonetheless, undesirable because it is associated with an increased likelihood of the droplets coalescing and the emulsion breaking. Other additives include buffers, antioxidants, and preservatives. Emulsions for oral administration are usually oil (the active ingredient) in water, and facilitate the administration of oily substances such as castor oil or liquid paraffin in a more palatable form.

A paste is a 2-component semi-solid in which drug is dispersed as a powder in an aqueous or fatty base. The particle size of the active ingredient in pastes can be as large as 100 μm. The vehicle containing the drug may be water; a polyhydroxy liquid such as glycerin, propylene glycol, or polyethylene glycol; a vegetable oil; or a mineral oil. Other formulation excipients include thickening agents, cosolvents, adsorbents, humectants, and preservatives. The thickening agent may be a naturally occurring material such as acacia or tragacanth, or a synthetic or chemically modified derivative such as xanthum gum or hydroxypropylmethyl cellulose. The degree of cohesiveness, plasticity, and syringeability of pastes is attributed to the thickening agent. It may be necessary to include a cosolvent to increase the solubility of the drug. Syneresis of pastes is a form of instability in which the solid and liquid components of the formulation separate over time; it is prevented by including an adsorbent such as microcrystalline cellulose. A humectant (eg, glycerin or propylene glycol) is used to prevent the paste that collects at the nozzle of the dispenser from forming a hard crust. Microbial growth in the formulation is inhibited using a preservative. It is critical that pastes have a pleasant taste or are tasteless.

A tablet consists of one or more active ingredients and numerous excipients and may be a conventional tablet that is swallowed whole, a chewable tablet, or a modified-release tablet (more commonly referred to as a modified-release bolus due to its large unit size). Conventional and chewable tablets are used to administer drugs to dogs and cats, whereas modified-release boluses are administered to cattle, sheep, and goats. The physical and chemical stability of tablets is generally better than that of liquid dosage forms. The main disadvantages of tablets are the bioavailability of poorly water-soluble drugs or poorly absorbed drugs, and the local irritation of the GI mucosa that some drugs may cause.

A capsule is an oral dosage form usually made from gelatin and filled with an active ingredient and excipients. Two common capsule types are available: hard gelatin capsules for solid-fill formulations, and soft gelatin capsules for liquid-fill or semi-solid-fill formulations. Soft gelatin capsules are suitable for formulating poorly water-soluble drugs because they afford good drug release and absorption by the GI tract. Gelatin capsules are frequently more expensive than tablets but have some advantages. For example, particle size is rarely altered during capsule manufacture, and capsules mask the taste and odor of the active ingredient and protect photolabile ingredients.

A powder is a formulation in which a drug powder is mixed with other powdered excipients to produce a final product for oral administration. Powders have better chemical stability than liquids and dissolve faster than tablets or capsules because disintegration is not an issue. This translates into faster absorption for those drugs characterized by dissolution rate-limited absorption. Unpleasant tastes can be more pronounced with powders than with other dosage forms and can be a particular concern with in-feed powders, in which it contributes to variable ingestion of the dose. Moreover, sick animals often eat less and are therefore not amenable to treatment with in-feed powder formulations. Drug powders are principally used prophylactically in feed, or formulated as a soluble powder for addition to drinking water or milk replacer. Powders have also been formulated with emulsifying agents to facilitate their administration as liquid drenches.

A granule is a dosage form consisting of powder particles that have been aggregated to form a larger mass, usually 2-4 mm in diameter. Granulation overcomes segregation of the different particle sizes during storage and/or dose administration, the latter being a potential source of inaccurate dosing. Granules and powders generally behave similarly; however, granules must deaggregate prior to dissolution and absorption.

A premix is a solid dosage form in which an active ingredient, such as a coccidiostat, production enhancer, or nutritional supplement, is formulated with excipients. Premix products are mixed homogeneously with feed at rates (when expressed on an active ingredient basis) that range from a few milligrams to ˜200 g/ton of food/beverage. The density, particle size, and geometry of the premix particles should match as closely as possible those of the feed in which the premix will be incorporated to facilitate uniform mixing. Issues such as instability, electrostatic charge, and hygroscopicity must also be addressed. The excipients present in premix formulations include carriers, liquid binders, diluents, anti-caking agents, and anti-dust agents. Carriers, such as wheat middlings, soybean mill run, and rice hulls, bind active ingredients to their surfaces and are important in attaining uniform mixing of the active ingredient. A liquid binding agent, such as a vegetable oil, should be included in the formulation whenever a carrier is used. Diluents increase the bulk of premix formulations, but unlike carriers, do not bind the active ingredients. Examples of diluents include ground limestone, dicalcium phosphate, dextrose, and kaolin. Caking in a premix formulation may be caused by hygroscopic ingredients and is addressed by adding small amounts of anti-caking agents such as calcium silicate, silicon dioxide, and hydrophobic starch. The dust associated with powdered premix formulations can have serious implications for both operator safety and economic losses, and is reduced by including a vegetable oil or light mineral oil in the formulation. An alternate approach to overcoming dust is to granulate the premix formulation.

A medicated block is a compressed feed material that contains an active ingredient, such as a drug, anthelmintic, surfactant (for bloat prevention), or a nutritional supplement, and is commonly packaged in a cardboard box. Ruminants typically have free access to the medicated block over several days, and variable consumption may be problematic. This concern is addressed by ensuring the active ingredient is nontoxic, stable, palatable, and preferably of low solubility. In addition, excipients in the formulation modulate consumption by altering the palatability and/or the hardness of the medicated block. For example, molasses increases palatability and sodium chloride decreases it. Additionally, the incorporation of a binder such as lignin sulfonate in blocks manufactured by compression or magnesium oxide in blocks manufactured by chemical reaction, increases hardness. The hygroscopic nature of molasses in a formulation may also impact the hardness of medicated blocks and is addressed by using appropriate packaging.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried cannabis extracts contained therein to be orally administered without drinking.

According to some embodiments of the present invention, the composition may comprise any suitable flavor or combination of flavors.

The composition may further comprise other additives, coloring, emulsifiers. The flavors and additives may be of a natural, semi-synthetic, synthetic source or combinations thereof.

In another embodiment of the present invention, the composition further comprises fructose, sorbitol, microcrystalline cellulose, magnesium stearate, or any combination thereof. In another embodiment, the composition further comprises chamomile. In another embodiment, the composition further comprises ginger. In another embodiment, the composition further comprises peppermint. In another embodiment, the composition further comprises anise. In another embodiment, the composition further comprises fennel. In another embodiment, the composition further comprises thyme. In another embodiment, the composition further comprises Arsenicum album. In another embodiment, the composition further comprises Carbo vegetabilis. In another embodiment, the composition further comprises Ignatia, homeopathic ipecac. In another embodiment, the composition further comprises Nux vomica. In another embodiment, the composition further comprises Zingiber officinale.

In another embodiment, the composition of the present invention is in the form of a chewing gum product. In another embodiment, chewing gum compositions contemplated by the present invention comprise all types of sugar and sugarless chewing gums and chewing gum formulations known to those skilled in the art, including regular and bubble gum types. In another embodiment, chewing gum compositions of the invention comprise a chewing gum base, a modifier, a bulking agent or sweetener, and one or more other additives such as, flavoring agents, colorants and antioxidants. In another embodiment, the modifying agents are used to soften, plasticize and/or compatibilize one or more of the components of the gum base and/or of the formulation as a whole.

In another embodiment, the present invention provides a soft, chewable dosage form which is pliable and chewy, yet dissolves quickly in the mouth, has a long shelf life, contains little moisture which improves stability and decreases the tendency for the dosage form to dry out, does not require cooking or heating as part of the manufacturing process. In another embodiment, the dosage form is used as a matrix for dried cannabis extracts.

In another embodiment, the chewable tablet of the invention comprises a metal salt such as calcium, magnesium, aluminum salt, or any mixture thereof. In another embodiment, the chewable tablet of the invention comprises hydroxyalkyl cellulose. In another embodiment, the chewable tablet of the invention comprises low viscosity hydroxyalkyl cellulose. In another embodiment, the chewable tablet of the invention comprises high viscosity hydroxyalkyl cellulose.

In another embodiment, the chewable tablet of the invention comprises various additives. In another embodiment, the chewable tablet of the invention comprises sweeteners. In another embodiment, the chewable tablet of the invention comprises acidic ingredients. In another embodiment, the chewable tablet of the invention comprises taste correctives. In another embodiment, the chewable tablet of the invention comprises polymeric compounds. In another embodiment, the chewable tablet of the invention comprises essential oils.

In another embodiment, the chewable tablet of the invention is a soft tablet. In another embodiment, the chewable tablet of the invention is made in a state of soft candy. In another embodiment, the chewable tablet of the invention is made in a state of jelly.

In another embodiment, the chewable tablet of the invention comprises a core comprising the vitamins of the invention. In another embodiment, the chewable tablet of the invention comprises an outer layer wrapping the core which is made up of chewable base such as a gum, a soft candy or a caramel.

In another embodiment, the compositions of the present invention may be provided in any suitable food of a solid, semi-solid or liquid form.

The preparation of pharmaceutical compositions that contain a dried cannabis extract, for example by mixing, granulating, or tablet-forming processes, is well understood in the art. The dried cannabis extracts are often mixed with excipients that are pharmaceutically acceptable and compatible with the active ingredient. For oral administration, the active ingredients of compositions of the present invention are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions.

In another embodiment, additional methods of administering the dried cannabis extracts, or compound(s) isolated therefrom, of the invention comprise injectable dosage forms. In another embodiment, the injectable is administered intraperitonealy. In another embodiment, the injectable is administered intramuscularly. In another embodiment, the injectable is administered intradermally. In another embodiment, the injectable is administered intravenously. Each possibility represents a separate embodiment of the present invention.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, additional methods of administering the dried cannabis extracts of the invention comprise dispersions, suspensions or emulsions. In another embodiment, the dispersion, suspension or emulsion is administered orally. In another embodiment, the solution is administered by infusion. In another embodiment, the solution is a solution for inhalation. Each possibility represents a separate embodiment of the present invention.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active compound agent over a period of time. Each possibility represents a separate embodiment of the present invention.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, the pharmaceutical compositions provided herein are controlled-release compositions, i.e. compositions in which the active compounds are released over a period of time after administration. Controlled- or sustained-release compositions include formulation in lipophilic depots (e.g. fatty acids, waxes, oils). In another embodiment, the composition is an immediate-release composition, i.e. a composition in which all the active compound is released immediately after administration. Each possibility represents a separate embodiment of the present invention.

In another embodiment, the pharmaceutical composition is delivered in a controlled release system. In another embodiment, the agents are administered using intravenous infusion, an implantable osmotic pump, a transdermal patch, liposomes, or other modes of administration. In another embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the therapeutic target, thus requiring only a fraction of the systemic dose (see, e.g., Goodson, in Medical Applications of Controlled Release, supra, vol. 2, pp. 115-138 (1984); and Langer R, Science 249: 1527-1533 (1990). Each possibility represents a separate embodiment of the present invention.

The compositions also include, in another embodiment, incorporation of the active materials into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Each possibility represents a separate embodiment of the present invention.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors. Each possibility represents a separate embodiment of the present invention.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications also increase, in another embodiment, the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound. Each possibility represents a separate embodiment of the present invention.

The compositions of the present invention may comprise one or more additional components may further include an additional component selected from the group consisting of an anti-static agent, a buffering agent, a bulking agent, a chelating agent, a colorant, a diluent, a dye, an emollient, a fragrance, an occlusive agent, a pH-adjusting agent, a preservative, and a vitamin

The compositions of the present invention may comprise one or more additional active agents, selected from the group consisting of active herbal extracts, analgesics, anti-allergic agents, anti-aging agents, anti-bacterials, antibiotic agents, anticancer agents, antidandruff agents, antidepressants, anti-dermatitis agents, anti-edemics, antihistamines, anti-helminths, anti-hyperkeratolyte agents, anti-inflammatory agents, anti-irritants, anti-microbials, anti-mycotics, anti-proliferative agents, antioxidants, anti-wrinkle agents, anti-pruritics, antiseptic agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, keratolytic agents, lactams, metals, metal oxides, mitocides, neuropeptides, non-steroidal anti-inflammatory agents, oxidizing agents, photodynamic therapy agents, retinoids, sanatives, scabicides, self-tanning agents, skin whitening agents, vasoconstrictors, vasodilators, vitamins, vitamin D derivatives and wound healing agents.

According to some embodiments, the composition may comprise one or more anti-oxidants/radical scavengers. The anti-oxidant/radical scavenger may be selected from butylated hydroxy benzoic acids and their salts, coenzyme Q10, coenzyme A, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to inducing remission. In another embodiment, “treating” refers to slowing the progression of a disease.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

1. Choi, M. and C. Lee, Immortalization of Primary Keratinocytes and Its Application to Skin Research. Biomol Ther (Seoul), 2015. 23(5): p. 391-9.

2. Kjaer, T. N., et al., Resveratrol ameliorates imiquimod-induced psoriasis-like skin inflammation in mice. PLoS One, 2015. 10(5): p. e0126599.

3. Jenkins, G., Molecular mechanisms of skin ageing. Mech Ageing Dev, 2002. 123(7): p. 801-10.

4. Rittie, L. and G. J. Fisher, UV-light-induced signal cascades and skin aging. Ageing Res Rev, 2002. 1(4): p. 705-20.

5. Zhang, S. and E. Duan, Fighting against Skin Aging: The Way from Bench to Bedside. Cell Transplant, 2018. 27(5): p. 729-738.

6. Aziz, J., et al., Molecular Mechanisms of Stress-Responsive Changes in Collagen and Elastin Networks in Skin. Skin Pharmacol Physiol, 2016. 29(4): p. 190-203.

7. Varani, J., et al., Decreased collagen production in chronologically aged skin: roles of age-dependent alteration in fibroblast function and defective mechanical stimulation. Am J Pathol, 2006. 168(6): p. 1861-8.

8. Khorsandi D, M. A., Nazari R, Abzadeh G, Borhani S, Rahimmalek M, Sabzi H, Ziamahmoudi N., Personalized Medicine: Regulation of Genes in Human Skin Ageing. Journal of Allergy & Therapy, 2016. 7: p. 6.

9. Carrino, D. A., J. M. Sorrell, and A. I. Caplan, Age-related changes in the proteoglycans of human skin. Arch Biochem Biophys, 2000. 373(1): p. 91-101.

10. Dos Santos, M., et al., Perlecan expression influences the keratin 15-positive cell population fate in the epidermis of aging skin. Aging (Albany N.Y.), 2016. 8(4): p. 751-68.

11. Petersen, J. W. and J. Y. Douglas, Tenascin-X, collagen, and Ehlers-Danlos syndrome: tenascin-X gene defects can protect against adverse cardiovascular events. Med Hypotheses, 2013. 81(3): p. 443-7.

1. A method for treating mammalian skin, the method comprising:
a) combining at least one marijuana cultivar and at least one hemp cultivar to form at least one Cannabis line;
b) extracting at least one compound from said at least one Cannabis line to form an extract; and
c) treating skin with at least one of said extract and said at least one compound in an effective amount to treat said skin.

2. A method according to claim 1, wherein said treating step induces modulation of gene expression in at least one of skin cells and skin tissue; and wherein said modulation of gene said expression results in a reduction of at least one of an aging state and a disease state of said skin.

3. A method according to claim 1, wherein said at least one Cannabis line is selected from the group consisting of a marijuana/marijuana hybrid line, hemp/hemp hybrid line and hemp/marijuana hybrid line.

4. A method according to claim 3, wherein said at least one line is selected from the group consisting of designated line #4,#6, #8, #12, #13, #14, #15, #18, #5, #31, #39, #49, #69, #114, #166, #267, #273, #10, #11, #156, #155, #207 .

5. A method according to claim 1, wherein said extracting step comprises extracting flowers of said at least one Cannabis line.

6. A method according to claim 5, wherein said extracting step comprises extracting said at least one compound in at least one organic solvent.

7. A method according to claim 6, wherein said extracting step is performed at a temperature in the range of 15-60° C. and at a pressure in a range of 0.5-1.5 bar and wherein said at least one organic solvent comprises ethyl acetate.

8. A method according to claim 1, further comprising:
i. exposing said skin to UV radiation prior to said treating step

9. A method according to claim 8, wherein said modulation of gene expression results in a reduction of at least one of a photo-aging state and said disease state in said skin, and wherein said skin comprises at least one of skin cells and skin tissue.

10. A method according to claim 1, wherein said at least one compound is provided in a concentration in a range of 0.0001-0.05 μg/μl, 0.001-0.05 μg/μl, 0.001-0.005 μg/μl, 0.003-0.03 μg/μl or 0.007-0.015 μg/μl.

11. A method according to claim 1, wherein said at least one compound is provided in a solvent extract and said solvent extract exhibits skin healing properties.

12. A method according to claim 11, wherein said solvent extract is at least 2-20, 3-15, 4-12, 5-10 or 6-9 times as effective as at least one of THC and CBD, administered at the same concentration in treating said disease.

13. The method of claim 2, wherein the disease state is skin cancer.

14. The method of claim 13, wherein the skin cancer is non-melanoma skin cancer.

15. The method of claim 14, wherein the non-melanoma skin cancer is a squamous cell carcinoma or basal cell carcinoma.

16. The method of claim 2, wherein the disease state is an inflammatory skin disease.

17. The method of claim 2, wherein the disease state is psoriasis or atopic dermatitis or contact dermatitis or UV or other environmental factor-induced inflammation.

18. An organic extract of at least one plant line, said at least one plant line formed from combining at least one of:
a) at least one marijuana cultivar; and
b) at least one hemp cultivar,
wherein said organic extract comprises at least one compound suitable for treating a mammalian skin disease or disorder.

19. An organic extract according to claim 18, wherein said at least one plant line comprises a Cannabis sativa line.

20. An organic extract according to claim 18, wherein said mammalian skin disease or disorder is selected from the group consisting of skin cancer, non-melanoma skin cancer, squamous cell carcinoma, basal cell carcinoma, cancer, an inflammatory skin disease, psoriasis, atopic dermatitis, contact dermatitis, a UV-induced disorder, a burn, a cut, a scar, a skin insult, or other environmental factor-induced inflammation.
